(19) United States
(12) Patent Application Publication
(10) Pub. No.: US 2002/0045184 A1
(43) Pub. Date: Apr. 18, 2002

(54) Packaging system

(76) Inventor: Chih-Ming Chen, Davie, FL (US)

Correspondence Address: DAVIDSON, DAVIDSON & KAPPEL, LLC, 485 SEVENTH AVENUE, 14TH FLOOR, NEW YORK, NY, 10018, US

(21) Appl. No.: 09/970,049

(22) Filed: Oct. 2, 2001

Related U.S. Application Data
(60) Provisional application No. 60/237,220, filed on Oct. 2, 2000.

(51) Int. Cl. C12Q 1/68

(52) U.S. Cl. 435/6; 206/532; 206/534

(57) ABSTRACT

In certain embodiments, the invention is directed to a package for dispensing a combination of a proton pump inhibitor and a non-steroidal anti-inflammatory drug.

This application claims priority from U.S. Provisional Application No. 60/237,220, filed Oct. 2, 2000, hereby incorporated by reference.

### BACKGROUND OF THE INVENTION

Nonsteroidal anti-inflammatory drugs (NSAIDS) are widely administered for the treatment of a variety of conditions including rheumatoid arthritis, osteoarthritis, juvenile arthritis, ankylosing spondylitis, tendinitis, bursitis, and gout. Despite the considerable therapeutic success that has been realized with these drugs, their use is limited due to gastrointestinal toxicity. For example, many NSAIDS have been found to cause gastrointestinal bleeding, ulceration or perforation upon repeated administration.

Proton pump inhibitors are a class of antisecretory compounds that suppresses gastric acid secretion by inhibition of the H⁺/K⁺ ATPase enzyme system at the secretory surface of gastric parietal cells. This enzyme system is the acid (proton) pump within the gastric mucosa. Thus, proton pump inhibitors can be used to block the final step of acid production in the gastrointestinal tract, thereby reducing the gastrointestinal toxicity associated with NSAID administration. As a result, it may be beneficial to administer one or more proton pump inhibiting agents in combination with NSAID therapy, in order to minimize the unwanted gastrointestinal side effects associated with the NSAID therapy.

Unfortunately, therapies that require the administration of multiple therapeutic agents, in differing amounts, over extended periods of time, pose particular problems for packagers of medicine, for patients, and for medical personnel who administer medicines to patients. Such combination therapies often cause confusion regarding when or whether a given dosage has been administered. Thus, one can easily administer too many or too few doses in a given period of time, thereby reducing the efficacy of the medication or causing bodily damage.

To increase compliance and convenience and to reduce the confusion that is associated with a combination therapy that provides for the administration of both an NSAID and a proton pump inhibitor, it would be beneficial to have a single packaging system that would provide each agent for easy distribution and administration. There is currently a need for such a packaging system.

### SUMMARY OF THE INVENTION

Applicant has discovered that many of the packaging and dosing problems associated with a combination therapy that provides for the administration of both an NSAID and a proton pump inhibitor can be remedied using a drug packaging system that provides one or more unit dosage forms of a non-steroidal anti inflammatory drug and one or more unit dosage forms of a proton pump inhibiting drug in a single packaging material.

Accordingly, the invention provides a drug packaging system comprising packaging material comprising therein one or more unit dosage forms of a nonsteroidal anti-inflammatory drug and one or more unit dosage forms of a proton pump inhibiting drug.

The invention also provides a blister card comprising a plurality of perforated pieces, wherein each perforated piece comprises one or more blister layers and a rupturable substrate, wherein the rupturable substrate and the one or more blister layers are on opposed sides of the blister card; and wherein each blister layer comprises therein one or more unit dosage forms of a nonsteroidal anti-inflammatory drug, one or more unit dosage forms of a proton pump inhibiting drug, or a combination thereof.

The invention also provides a kit comprising a container comprising therein a plurality of blister cards of the invention.

In other embodiments, the invention is directed to a drug packaging system as disclosed herein comprising packaging material comprising therein combined prescription drug therapy comprising one or more unit dosage forms of a first drug and one or more unit dosage forms of a second drug. Preferably, the first and second drug are independently selected from the group consisting of non-steroidal anti-inflammatory drugs, proton pump inhibitors, calcium channel blockers, angiotensin converting enzyme (ACE) inhibitors, anti-depressants, selective serotonin reuptake inhibitors, antihistamines, decongestants, biguanides, sulfonylureas, 3-hydroxy-3-methylglutaryl coenzyme A (HMG CoA) reductase inhibitors, anti-epileptic, and anti diabetics. It is meant that the two drugs can be the same drug, e.g., the same strength or different strengths.

In other embodiments, the invention is directed to a drug packaging system comprising packaging material comprising therein combined prescription drug therapy comprising one or more unit dosage forms of a first drug and one or more unit dosage forms of a second drug, wherein at least one of said first or second drug are selected from the group consisting of non-steroidal anti-inflammatory drugs, calcium channel blockers, angiotensin converting enzyme (ACE) inhibitors, anti-depressants, selective serotonin reuptake inhibitors, antihistamines, decongestants, biguanides, sulfonylureas, 3-hydroxy-3-methylglutaryl coenzyme A (HMG CoA) reductase inhibitors, anti-epileptic, and anti diabetics.

In other embodiments, the invention is directed to a drug packaging system comprising packaging material comprising therein combined prescription drug therapy comprising one or more unit dosage forms of a first drug and one or more unit dosage forms of a second drug, wherein said first and second drug are independently selected from the group consisting of antibiotics and anti-ulcer agents selected from the group consisting of H2 antagonists, antacids, bismuth compounds, prostaglandins, carbenoxolone and anticholinergic agents.

The drugs disclosed above and throughout the application include the base drug and pharmaceutically acceptable salts thereof.

### BRIEF DESCRIPTION OF THE FIGURES

FIG. 1 illustrates a preferred packaging material of the present invention (front view).

FIG. 2 illustrates a preferred packaging material of the present invention (rear view).

FIG. 3 illustrates a preferred packaging material of the present invention (front view).

FIG. 4 illustrates a preferred packaging material of the present invention (rear view).

FIG. 5 illustrates a preferred packaging material of the present invention (front view), wherein the period of administration is four weeks.

FIG. 6 illustrates a preferred packaging material of the present invention (rear view), wherein the period of administration is four weeks.

FIG. 7 illustrates a preferred packaging material of the present invention (front view), wherein the period of administration is two weeks.

FIG. 8 illustrates a preferred packaging material of the present invention (rear view), wherein the period of administration is two weeks.

FIG. 9 illustrates a preferred blister card of the present invention (front view) wherein the blister card is round.

FIG. 10 illustrates a preferred blister card of the present invention (rear view) wherein the blister card is round.

FIG. 11 illustrates a preferred blister card of the present invention (front view) wherein the blister card is round.

FIG. 12 illustrates a preferred blister card of the present invention (rear view) wherein the blister card is round.

FIG. 13 illustrates a preferred blister card of the present invention (front view) wherein the blister card is round and the period of administration is one month.

FIG. 14 illustrates a preferred blister card of the present invention (rear view) wherein the blister card is round and the period of administration is one month.

FIG. 15 illustrates a preferred blister card of the present invention (front view) wherein the blister card is round and the period of administration is one month.

FIG. 16 illustrates a preferred blister card of the present invention (rear view) wherein the blister card is round and the period of administration is one month.

FIG. 17 illustrates a preferred packaging material of the present invention (front view).

FIG. 18 illustrates a preferred packaging material of the present invention (rear view).

FIG. 19 illustrates a preferred packaging material of the present invention (front view).

FIG. 20 illustrates a preferred packaging material of the present invention (rear view).

FIG. 21 illustrates a preferred blister card of the present invention (front view) wherein the blister card includes perforations.

FIG. 22 illustrates a preferred blister card of the present invention (rear view) wherein the blister card includes perforations.

FIG. 23 illustrates a preferred blister card of the present invention (front view) wherein the blister card includes perforations.

FIG. 24 illustrates a preferred blister card of the present invention (rear view) wherein the blister card includes perforations.

FIG. 25 illustrates a preferred blister card of the present invention (side view) wherein the blister card includes a backing.

FIGS. 26-52 illustrate preferred embodiments of the present invention.

FIG. 53 illustrates a blister pack rack of the present invention.

### DETAILED DESCRIPTION OF THE INVENTION

Specific and preferred packaging materials, drug packaging systems, blister cards, kits, proton pump inhibitors, nonsteroidal anti-inflammatory drugs, and unit dosages described herein below are for illustration only; they do not exclude other packaging materials, proton pump inhibitors, nonsteroidal anti-inflammatory drugs, or unit dosages.

As used herein, the term “unit dosage form” means an administrable pharmaceutical composition comprising a discrete or measurable amount of an active agent in combination with a pharmaceutical carrier. For example, the term unit dosage form can include hard or soft gelatin capsules, cachets, or tablets, each containing a predetermined amount of an active agent as a powder or as granules. The term unit dosage form can also include lozenges comprising a predetermined amount of an active agent in a flavored base, such as sucrose and acacia or tragacanth. Unit dosage forms can be adapted to provide sustained release of an active ingredient, e.g., by combination thereof with certain hydrophilic polymer matrices, e.g., comprising natural gels, synthetic polymer gels or mixtures thereof, or using other sustained release technologies known in the art.

As used herein the term “indicia” includes marks, colors, symbols, letters, numbers, and the like, that provide information to aid with removal of medicaments from a packaging system of the invention, or that provide dosing information or instructions for a patient. For example, indicia can be included on unit dosage forms, blister layers, rupturable substrates, or backing layers, or on other packaging materials. Indicia can be printed, stamped, embossed, or embedded on the materials using techniques that are known in the packaging and printing field.

As used herein, the term “medicament” includes a unit dosage form of one or more proton pump inhibitors, a unit dosage form of one or more nonsteroidal anti-inflammatory drugs, or a combination thereof.

As used herein, the term container can include any structure that can enclose a plurality of packaging systems (e.g. blister cards) of the invention. Such containers typically facilitate the storage, transport, distribution or sale of the packaging systems of the invention. For example, suitable containers include cardboard or plastic boxes, as well as paper or plastic wrapping materials.

Packaging Materials

Any suitable packaging material can be employed in the packaging system of the invention, provided the unit dosage forms of the proton pump inhibitor and the nonsteroidal anti-inflammatory drug can be contained within the packaging material and are available for co-administration. Suitable packaging materials may include bottles, vials, boxes, foil wraps, and dispensing packs, such as those disclosed in U.S. Pat. Nos. 4,553,670; 5,954,204; 4,574,954; 4,850,489; 5,927,500; 4,158,411; 4,429,792; 3,211,503; 3,283,885; 3,311,229; 3,324,996; 3,380,578; 3,397,671; 3,494,322; 3,630,346; 3,759,371; 3,856,144; 4,211,326; 3,054,503; 3,503,493; 3,933,245; 4,371,080; 6,024,222; 2,012,405; 2,317,860; 3,324,995; 3,780,856; 3,835,995; 3,899,080; 2,012,405; 2,317,860; 3,324,995; 3,397,671; 3,494,322; 3,780,856; 3,835,995; 3,899,080; and 6,024,222; in U.S. Design Pat. No. 237 864; and references cited therein.

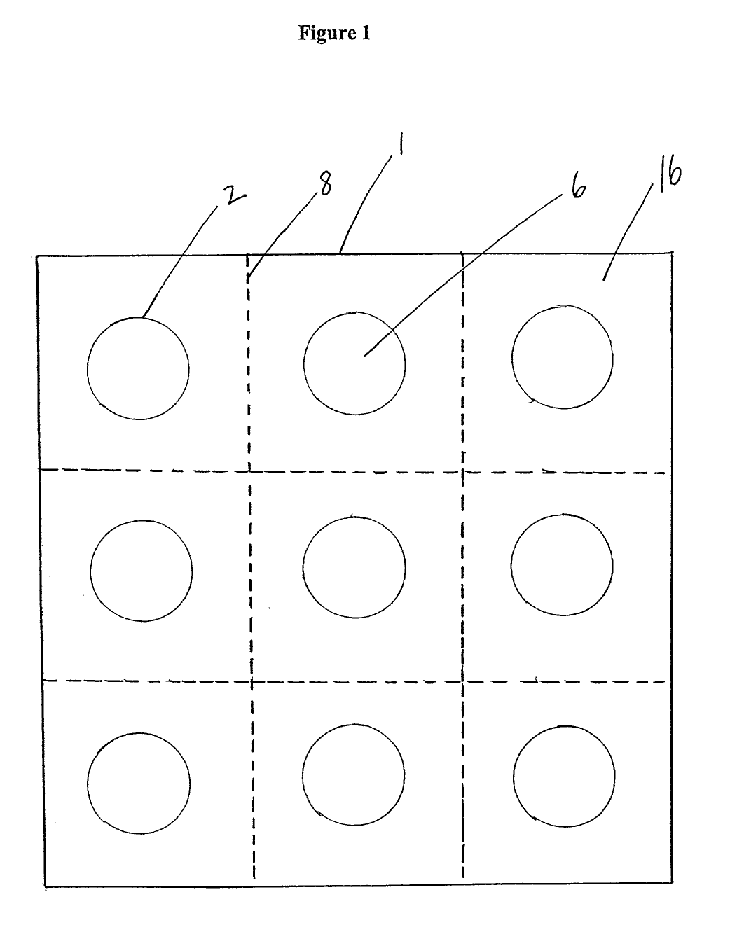

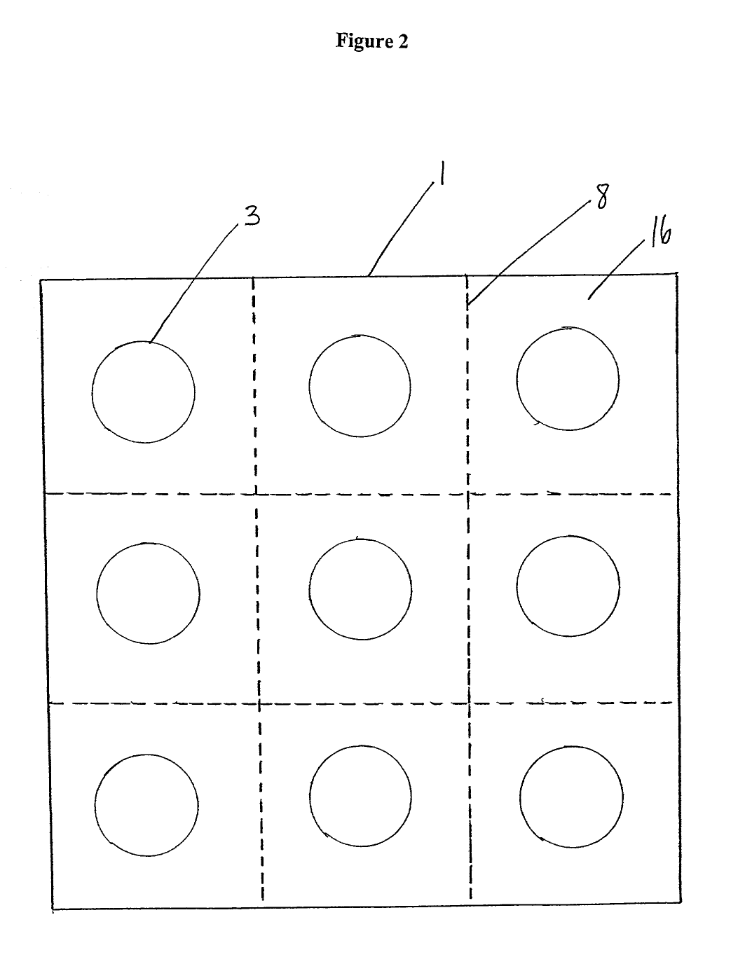

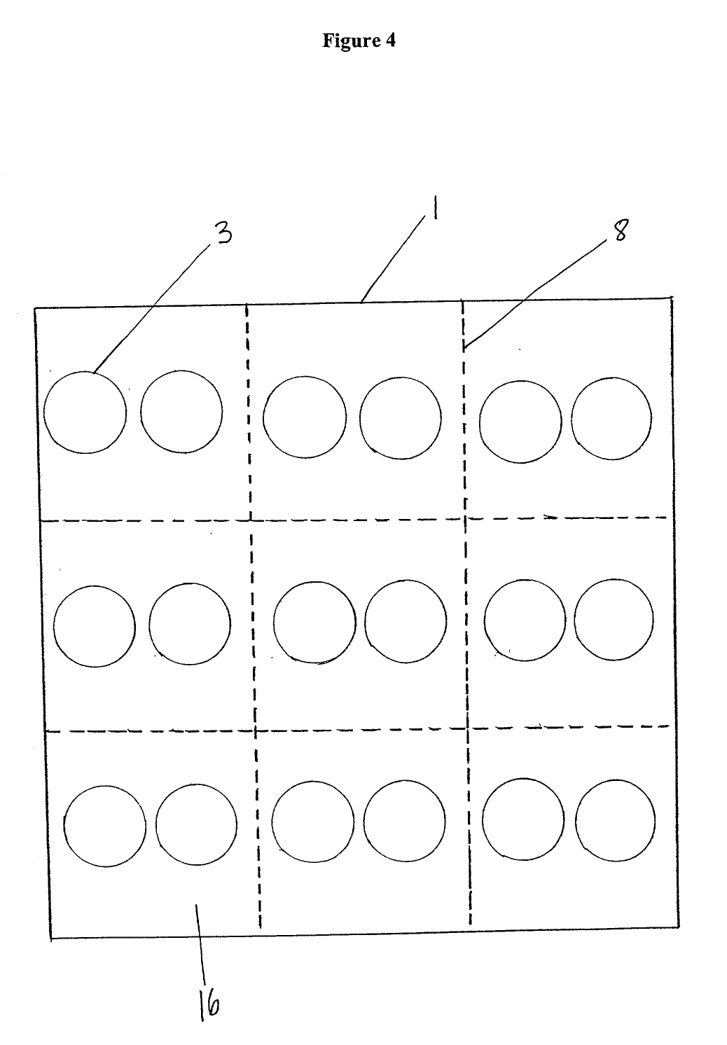

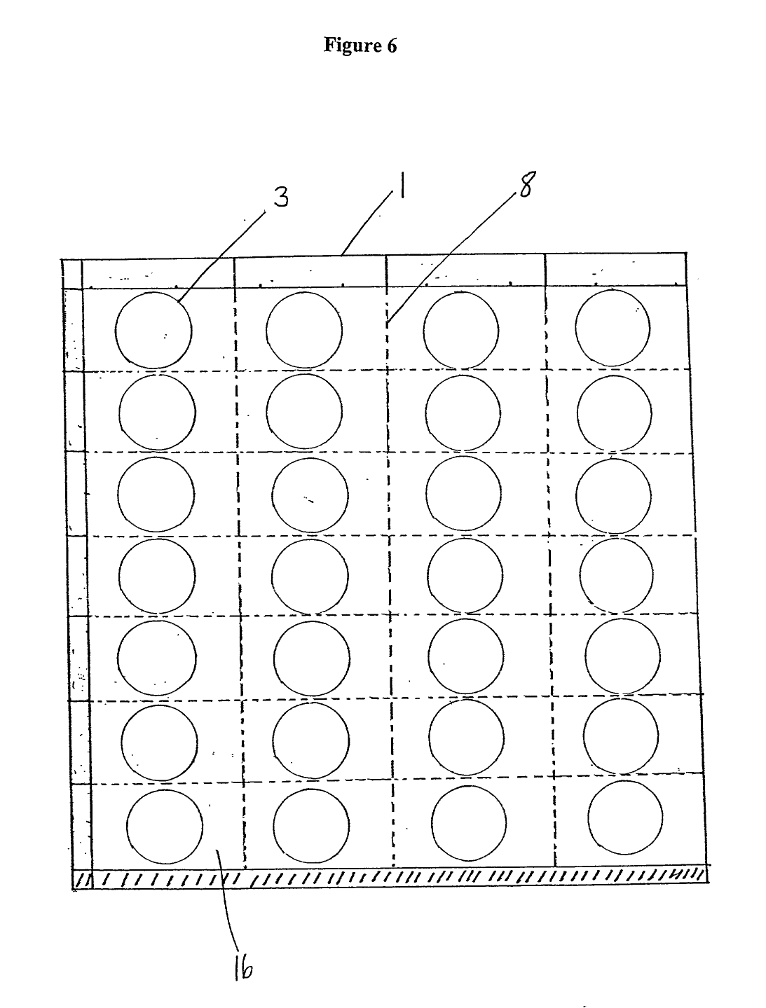

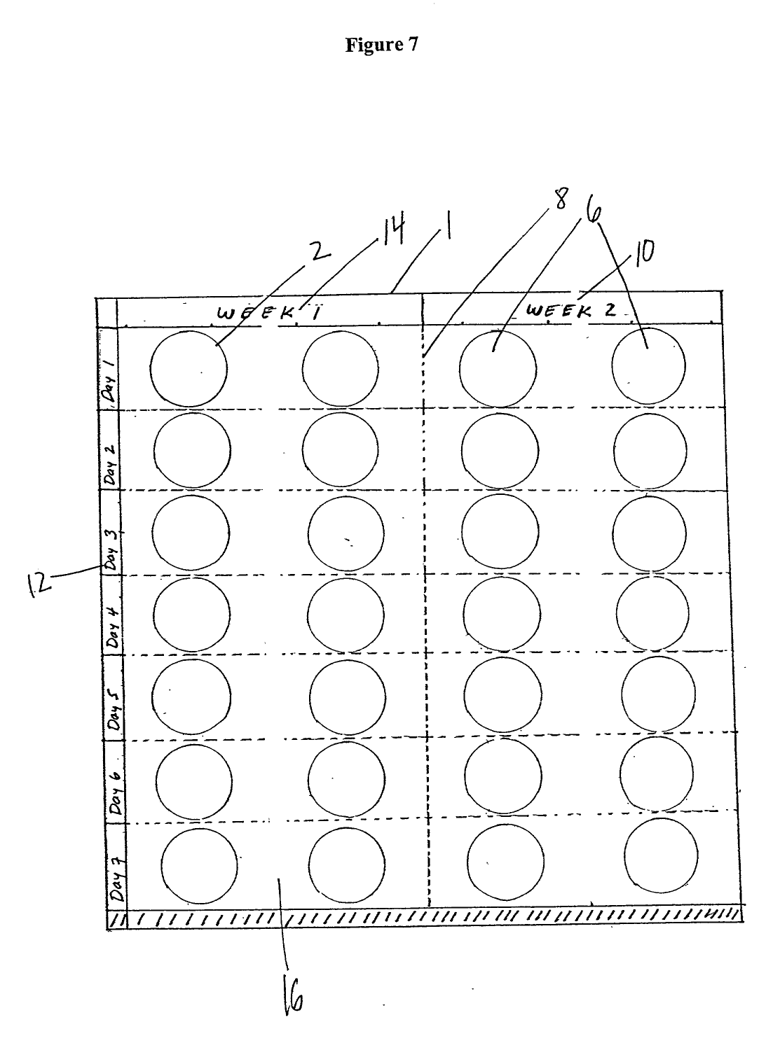

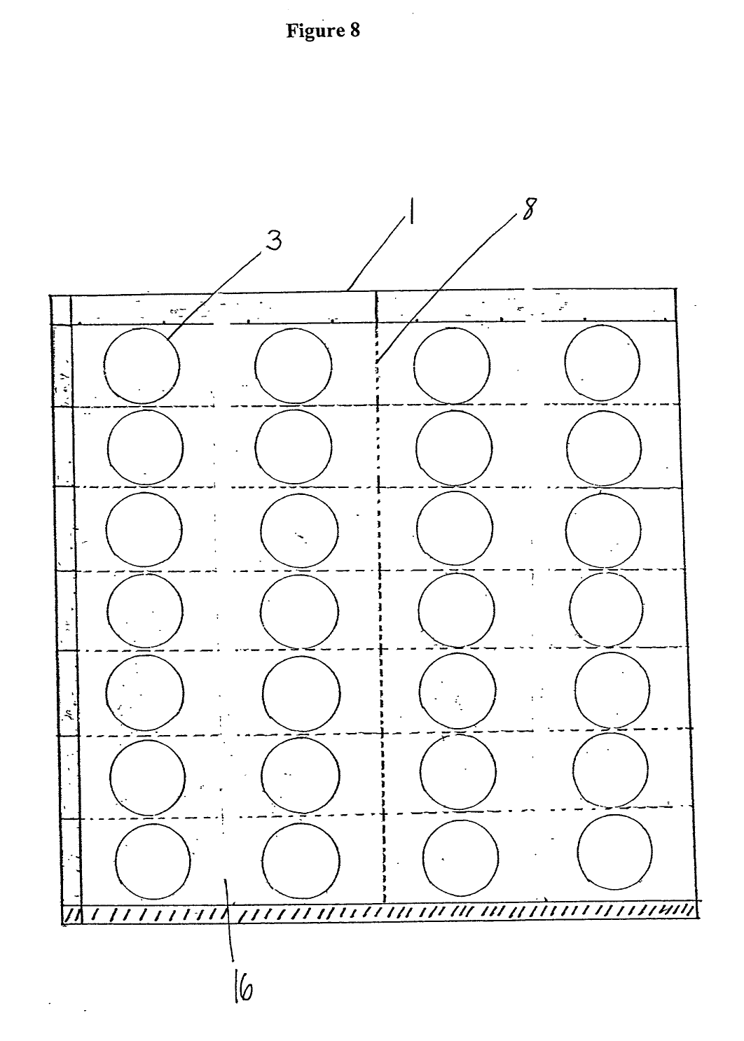

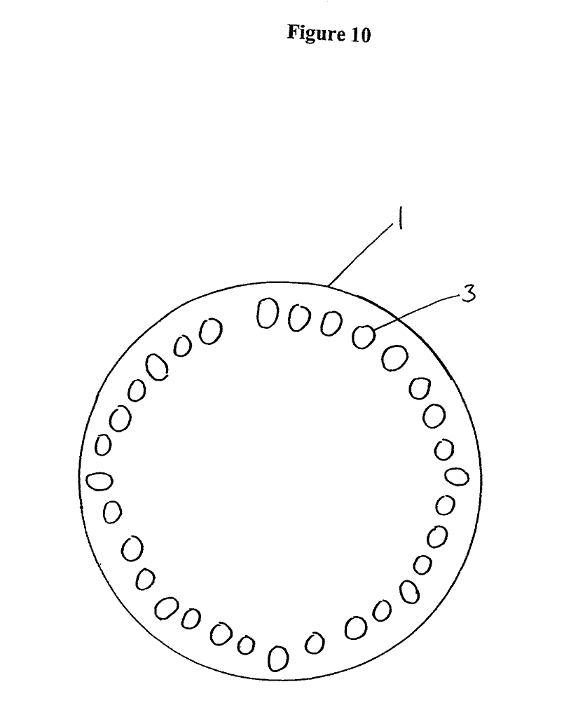

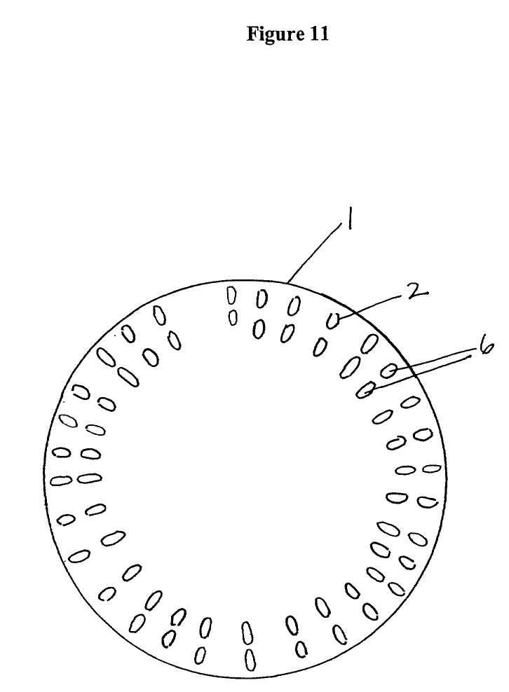

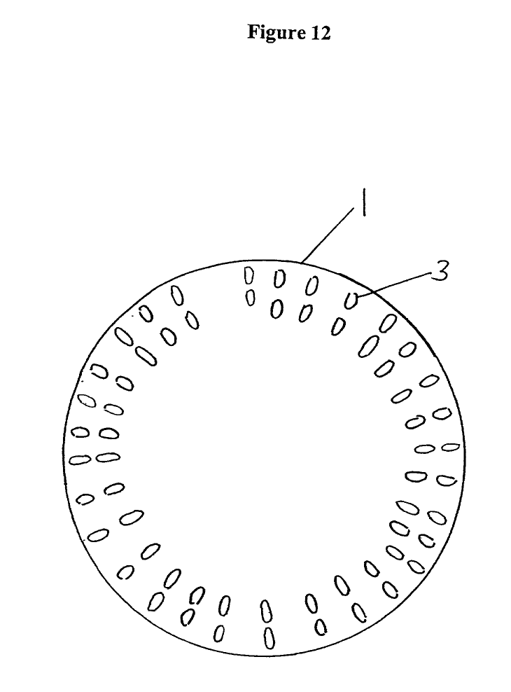

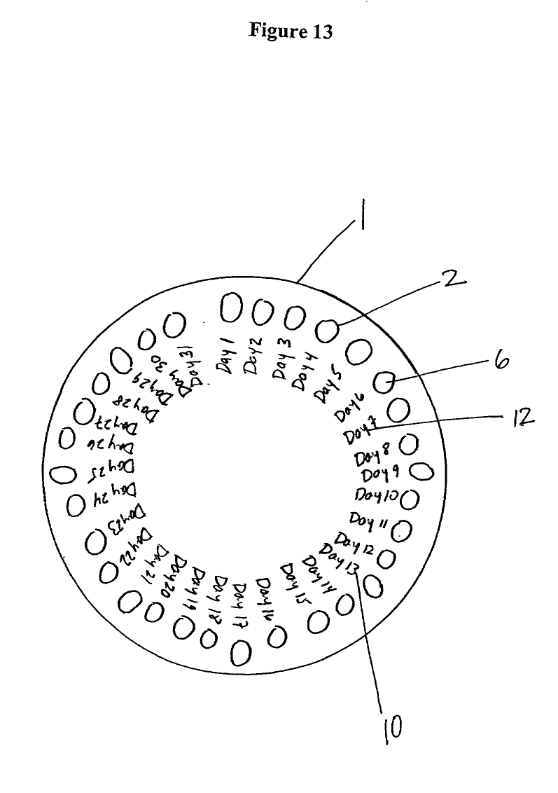

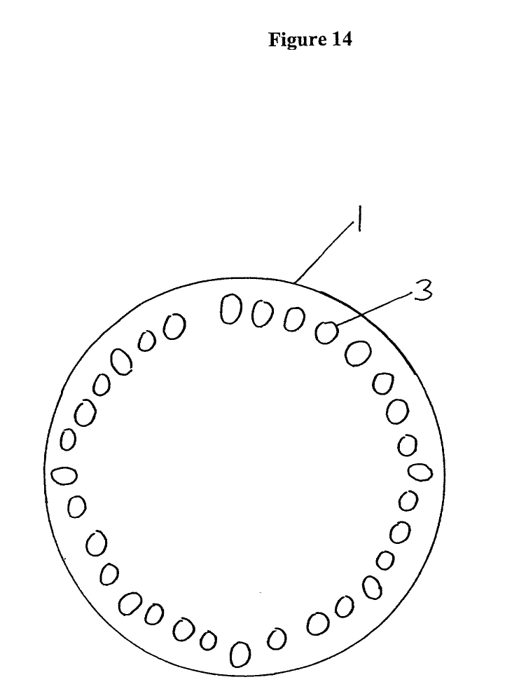

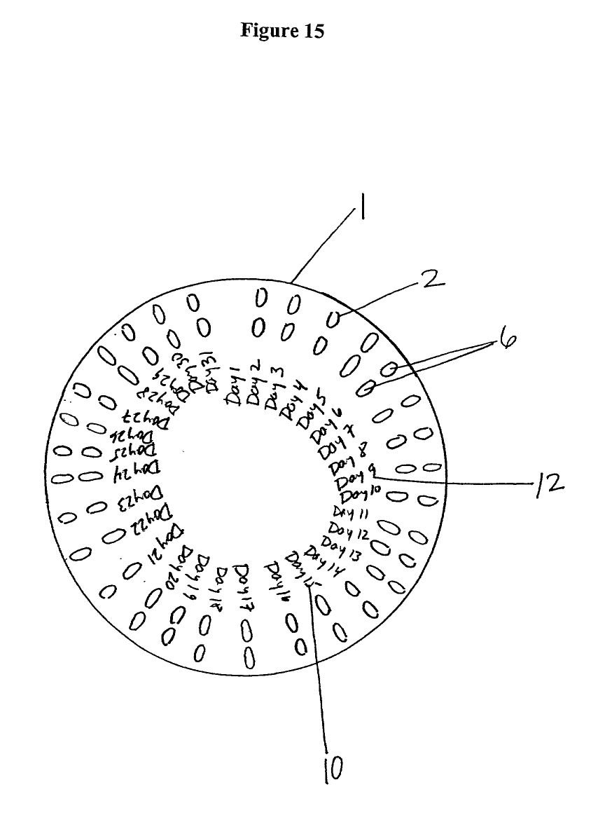

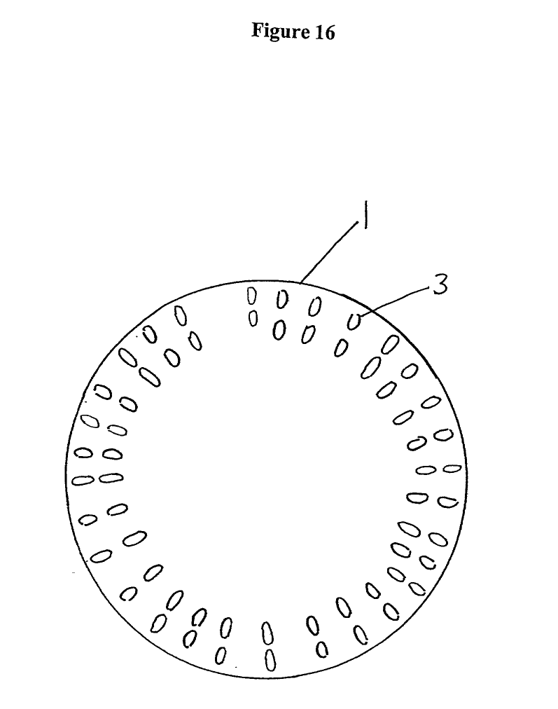

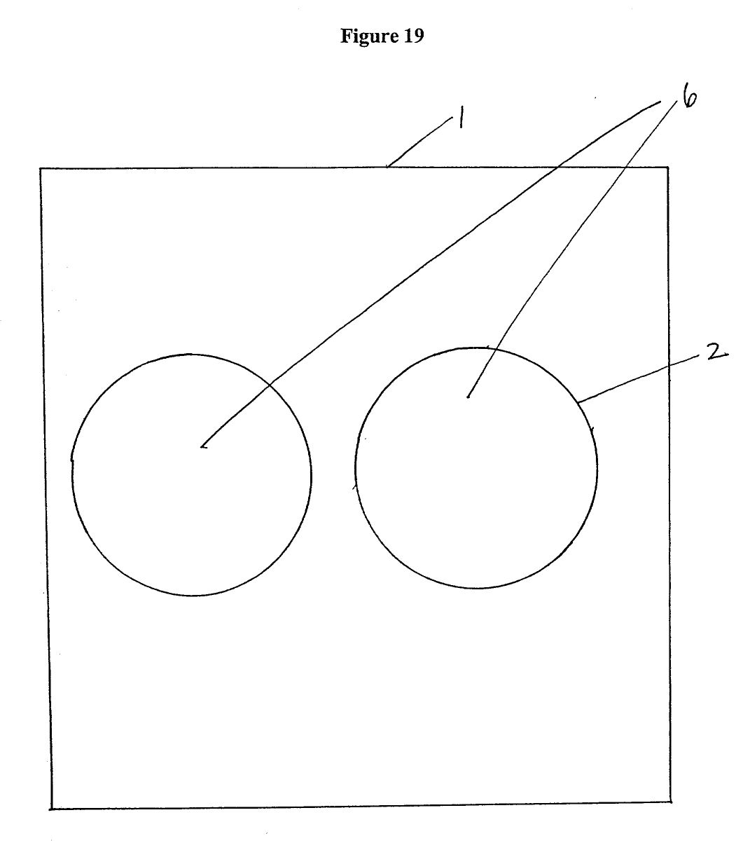

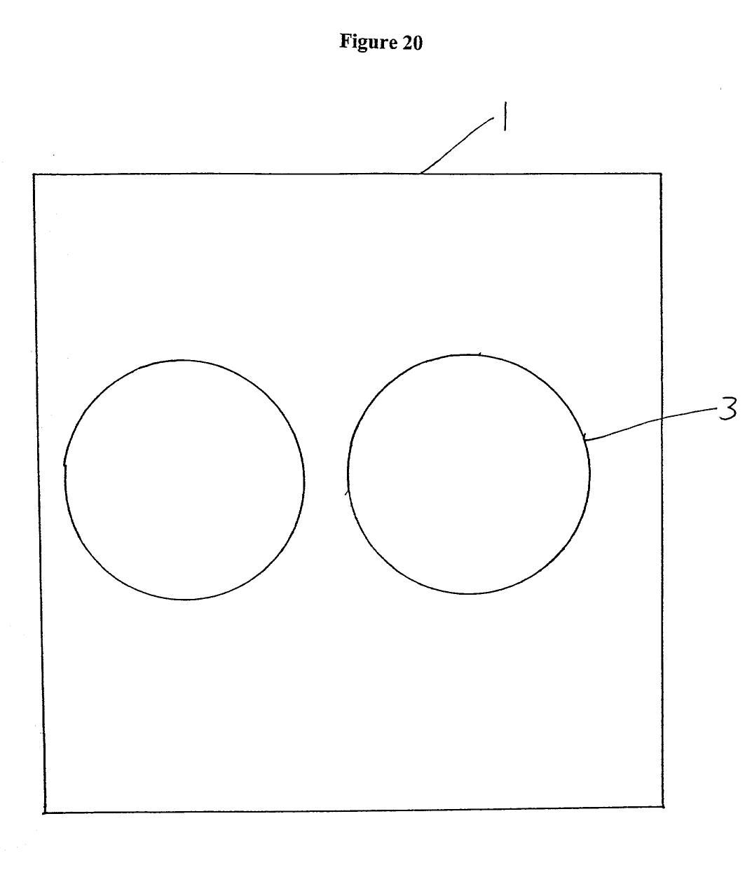

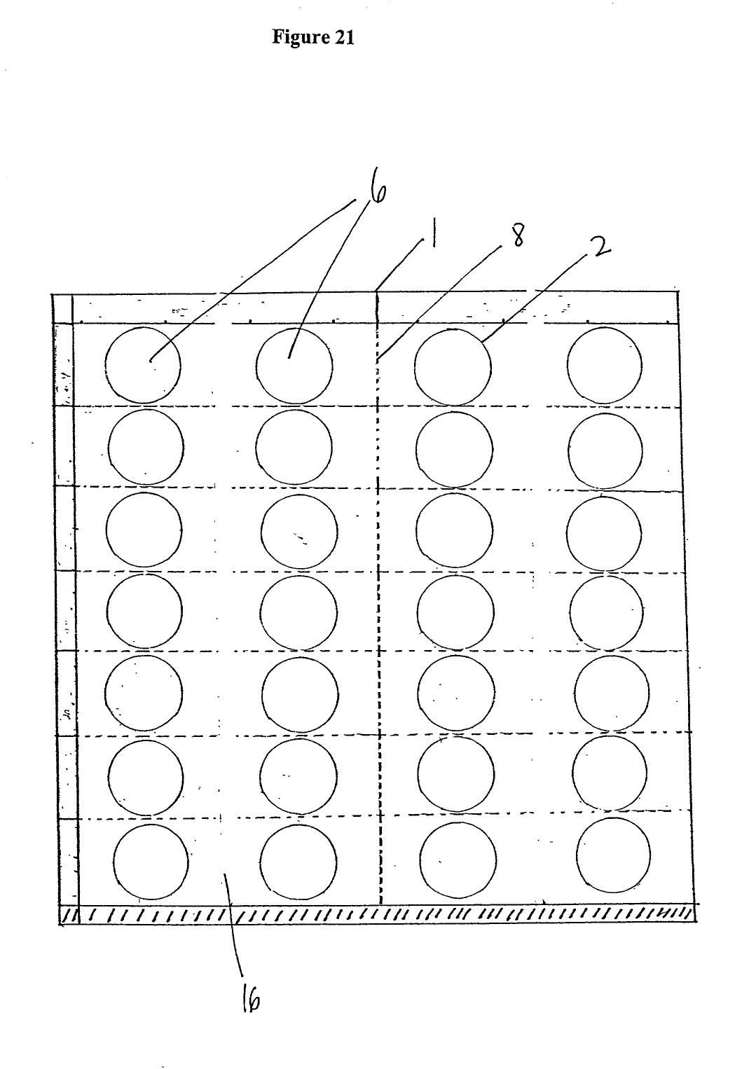

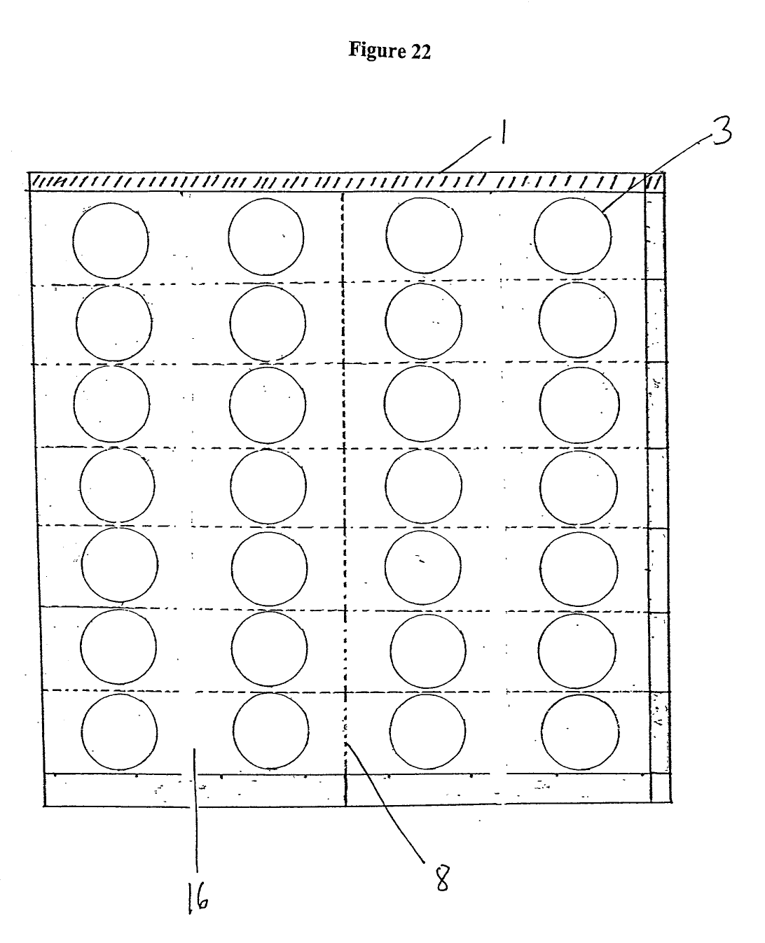

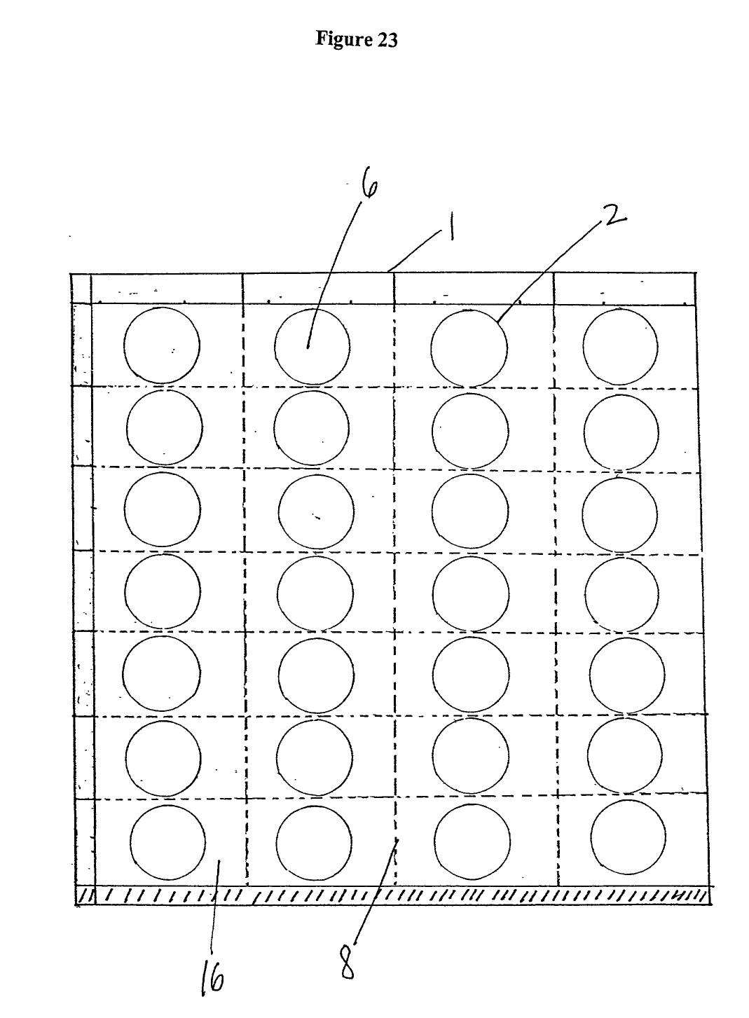

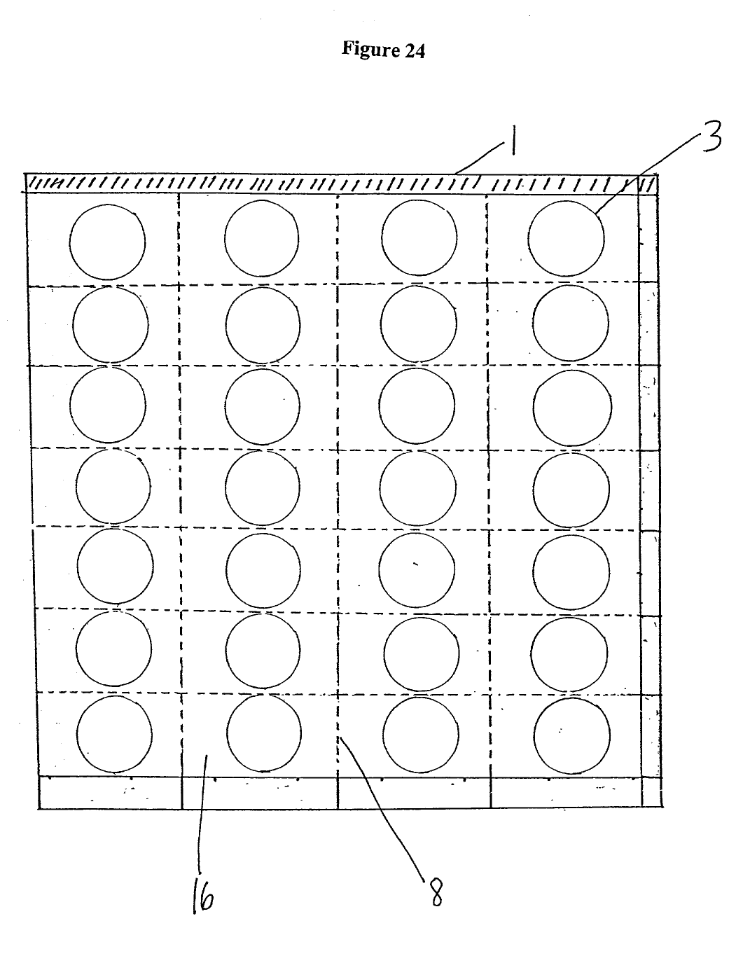

Numerous packaging materials are illustrated in the Figures. For example, referring to FIGS. 1-25, the packaging material can be a blister package 1 (e.g., a blister card). The blister package 1 includes one or more blister layers 2, a rupturable substrate 3 that is located opposite to the one or more blister layers 2, and a medicament 6 in the form of a tablet or pill can be contained between each of the one or more blister layers 2 and the rupturable substrate 3. The blister package 1 can also optionally includes backing 4 that is interposed between the one or more blister layers 2 and the rupturable substrate 3.

Each of the one or more blister layers 2 can be manufactured from any suitable material. Suitable exemplary materials include polyvinyl chloride, a thermoplastic material, a polyolefin, and combinations thereof.

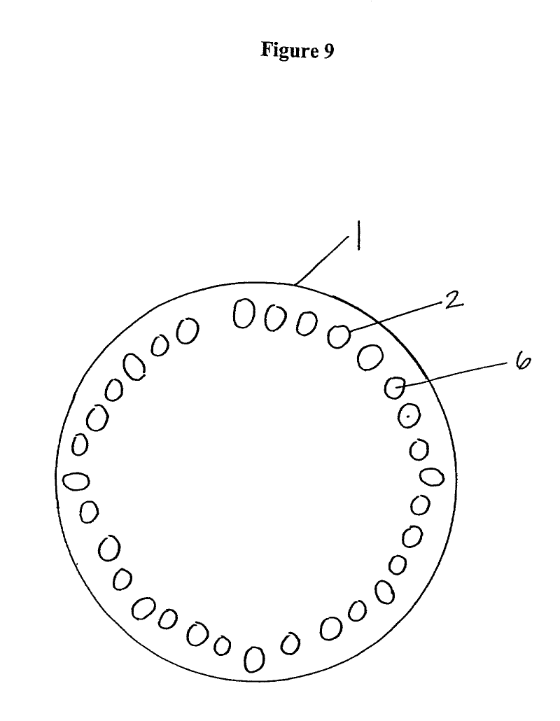

Each of the one or more blister layers 2 can have any suitable shape, provided each of the one or more blister layers 2 can contain therein the medicament 6. Suitable shapes include, e.g., circles (see, e.g., FIGS. 9-16), ovals, and rectangles (see, e.g., FIGS. 1-15 and 17-24).

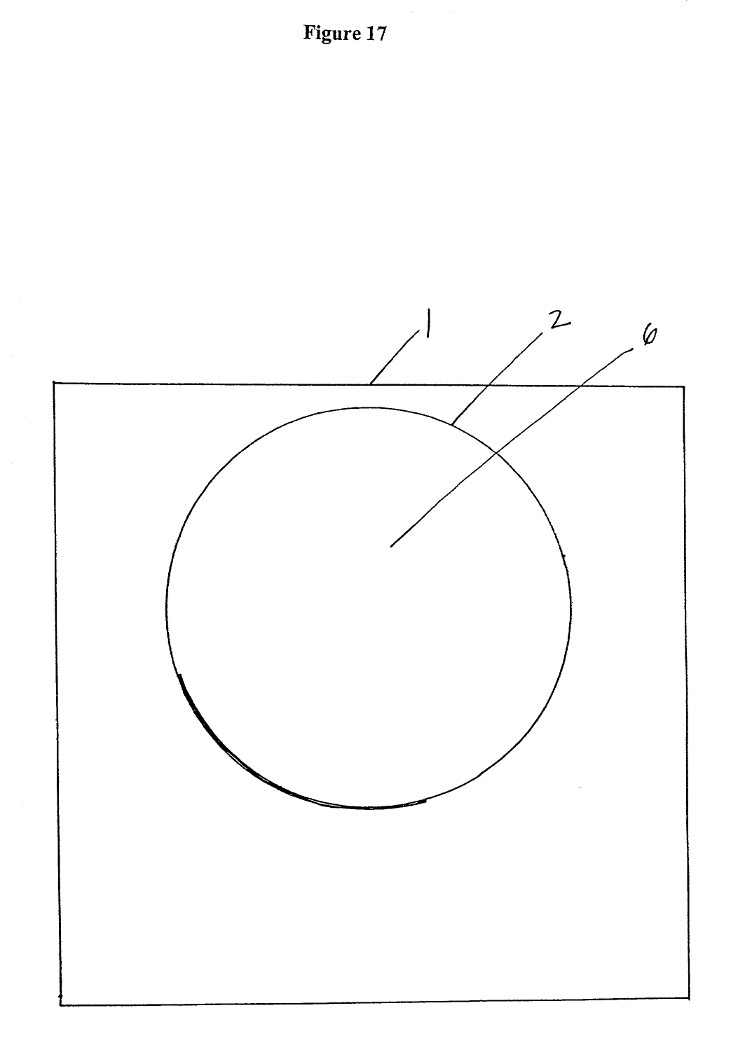

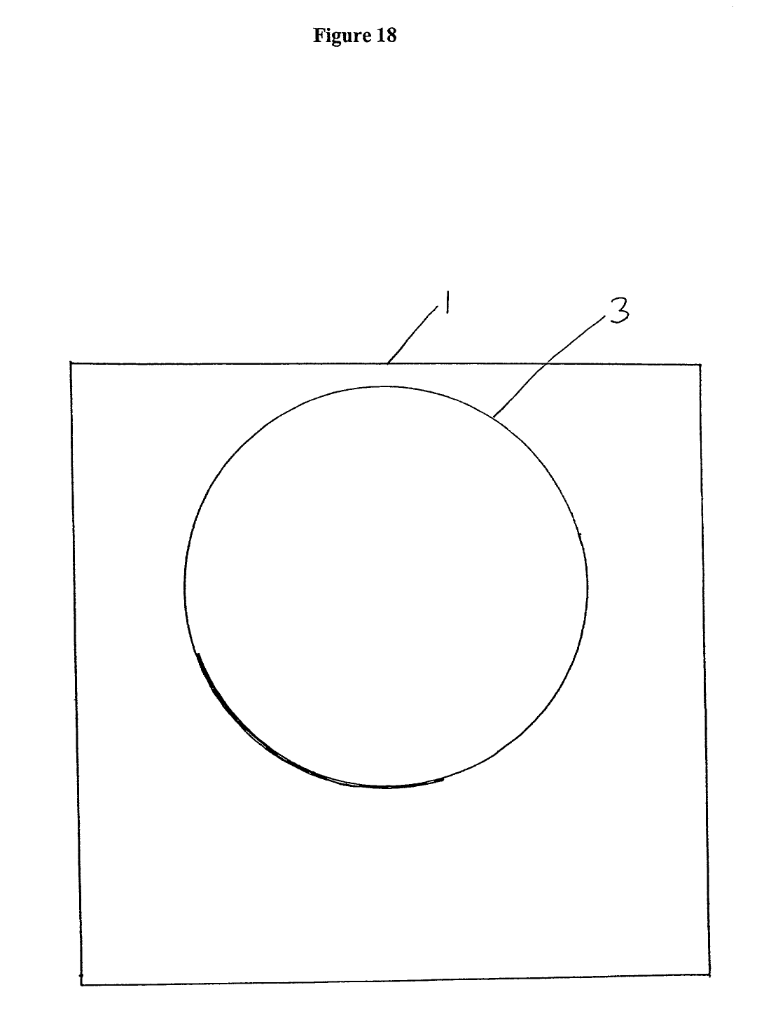

In one embodiment, the blister package 1 can include one blister layer 2, as shown in FIGS. 17-18. In such an embodiment, the blister layer 2 contains therein, one or more unit dosage forms of a proton pump inhibitor and one or more unit dosage forms of an NSAID. In another embodiment, the blister package 1 can include two or more blister layers 2, as shown in FIGS. 1-16 and 19-24. In such embodiment, the blister package 1 can preferably include about two blister layers 2 to about 120 blister layers 2, or about 7 blister layers 2 to about 31 blister layers 2. In such an embodiment, each of the two or more blister layers 2 can contain therein, one or more unit dosage forms of a proton pump inhibitor and/or one or more unit dosage forms of an NSAID.

Typically, the number of blister layers 2 present on a blister package 1 will be determined by the specific medicament 6 employed, as well as the course of administration for the medicament 6. For example, one proton pump inhibitor can be contained within a blister layer 2 and one NSA/D can be contained within another blister layer 2. Each of the proton pump inhibitor and the NSAID can be in the form of a daily dosage. Assuming the period of administration for such a combination therapy is one month, the number of blister layers 2 on the blister package 1 can conveniently be about 60 or about 62.

In another embodiment, one unit dosage form of a proton pump inhibitor can be contained within a blister layer 2, one unit dosage form of an NSAID can be contained within another blister layer 2, and a second unit dosage form of an NSAID can be contained within another (i.e., third) blister layer 2. Assuming the three unit dosage forms are to be taken daily and assuming the course of administration is one month, the number of blister layers 2 on the blister package 1 can conveniently be about 90 or about 96.

The rupturable substrate 3 can be manufactured from any suitable material. Suitable exemplary materials include tempered metal foil, paperboard, polyvinyl chloride, a polyolefin, polystyrene, a polyester, a fluoropolymer resin, and combinations thereof. The rupturable substrate 3 may be sealed to each of the one or more blister layers 2 or to the backing 4 through the application of heat and pressure as is typically done in the art through conventional thermal forming methods. The rupturable substrate 3 may also be composed of a plurality of laminated layers of different material (see for example U.S. Pat. No. 6,024,222), as long as the basic operation is not affected. The rupturable substrate 3 can optionally be replaced by a removable backing that can be peeled off to provide access to the medicament 6.

The rupturable substrate 3 can be a continuous surface that can essentially cover all the blister layers 2, or can be a plurality of surfaces each covering one or more blister layer 2. When the backing 4 is present, the rupturable substrate 3 can essentially cover the entire surface of the backing 4 or can cover a portion or portions of the backing 4, wherein each portion of the backing 4 corresponds to a blister layer 2. When the rupturable substrate 3 essentially covers the entire surface of the backing 4, the portion or portions of the rupturable substrate 3 that are located opposite to the one or more blister layers 2 will rupture upon dispensing the medicament 6. When the rupturable substrate 3 covers a portion or portions of the backing 4, the portion or portions will typically rupture upon dispensing the medicament 6.

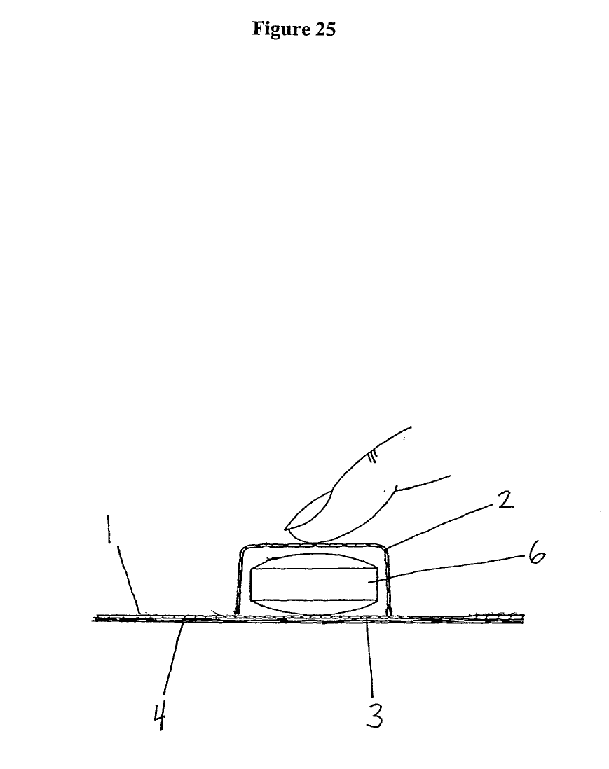

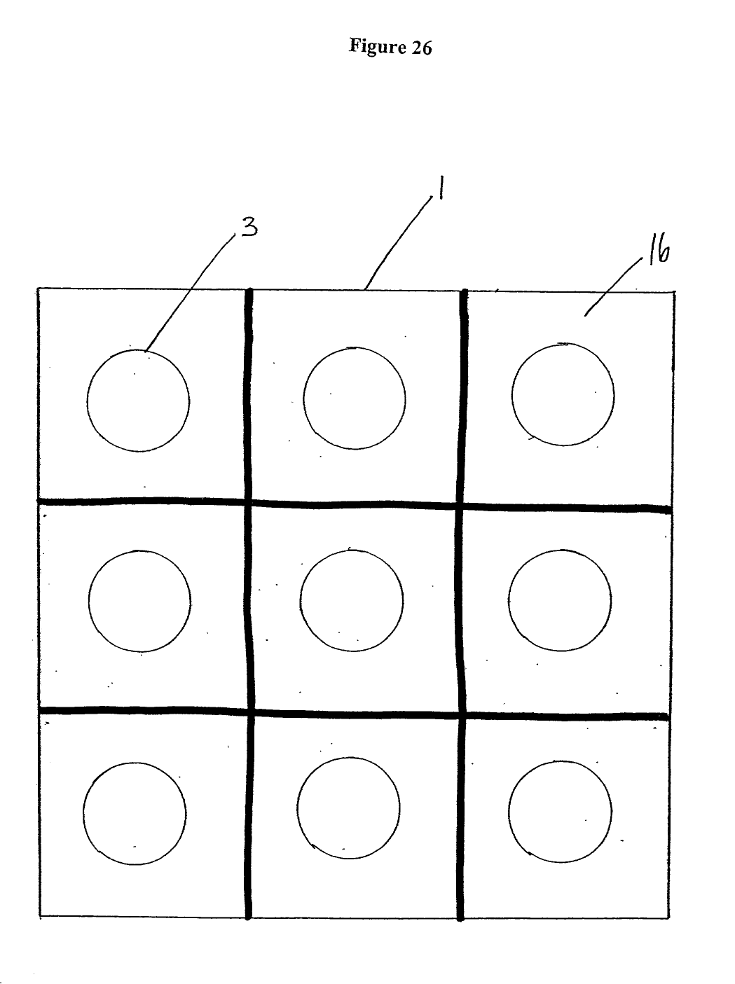

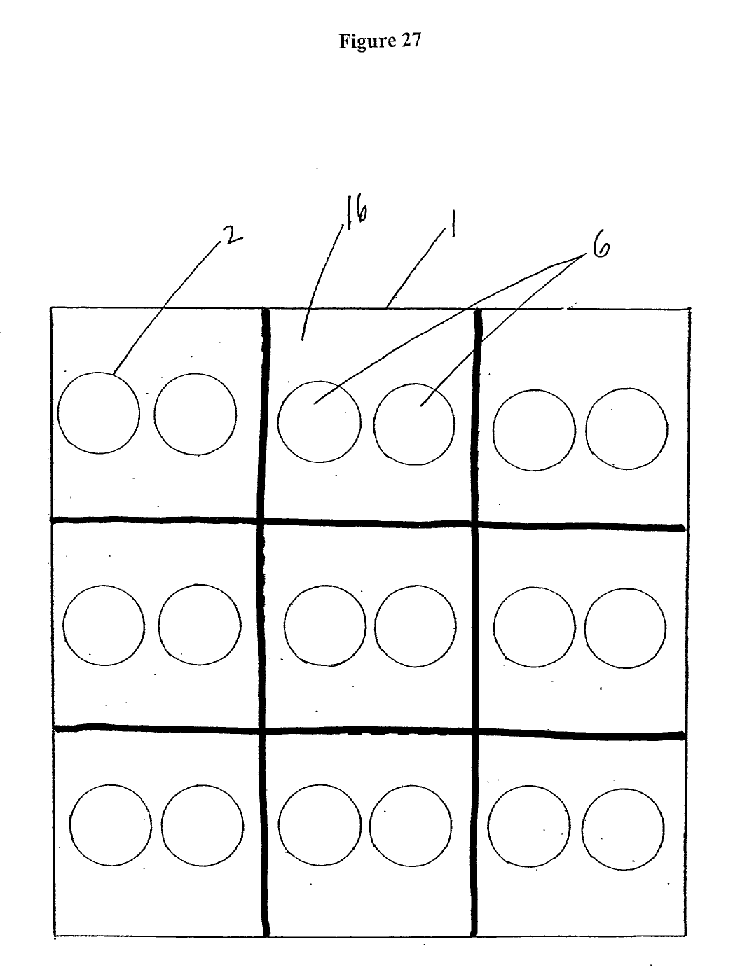

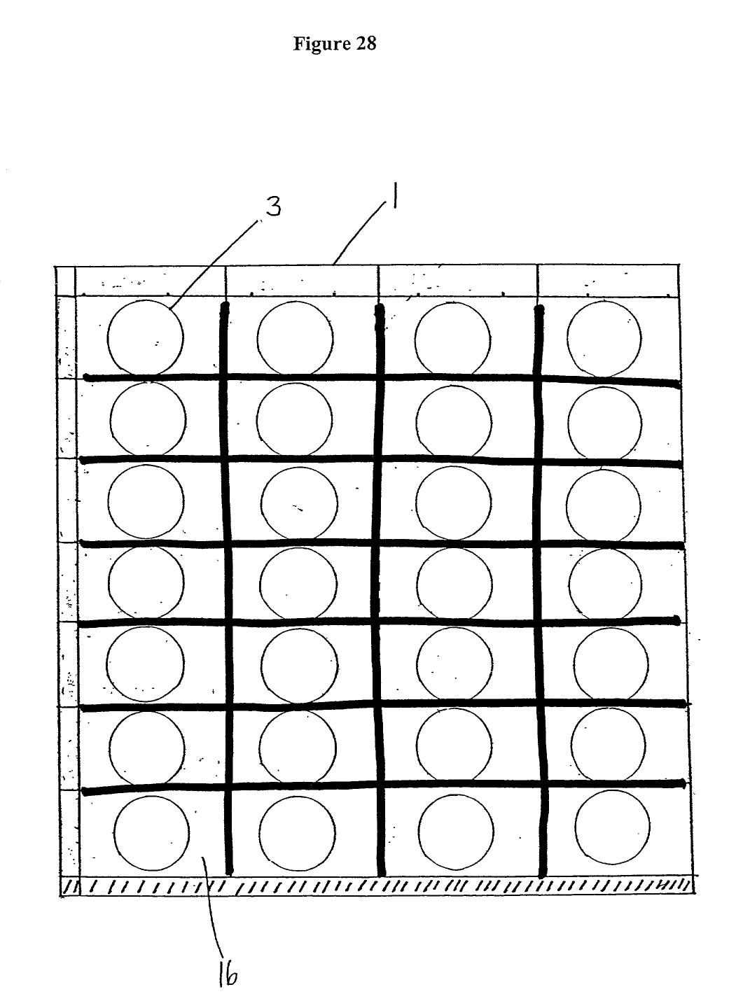

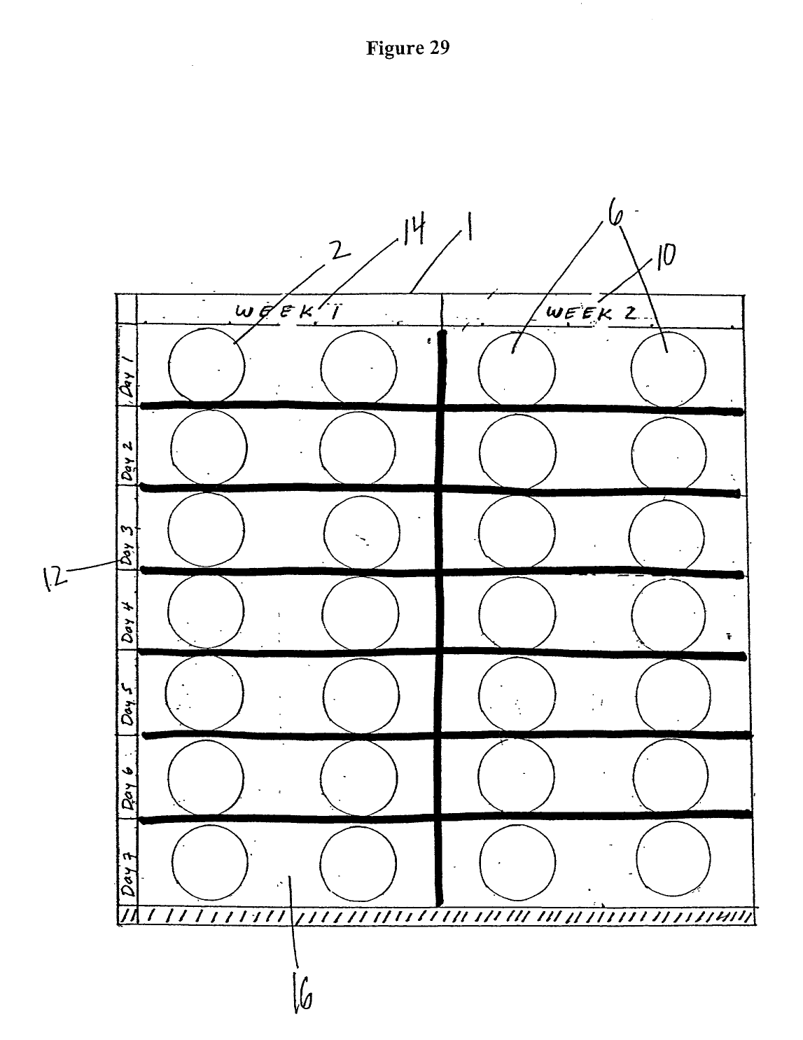

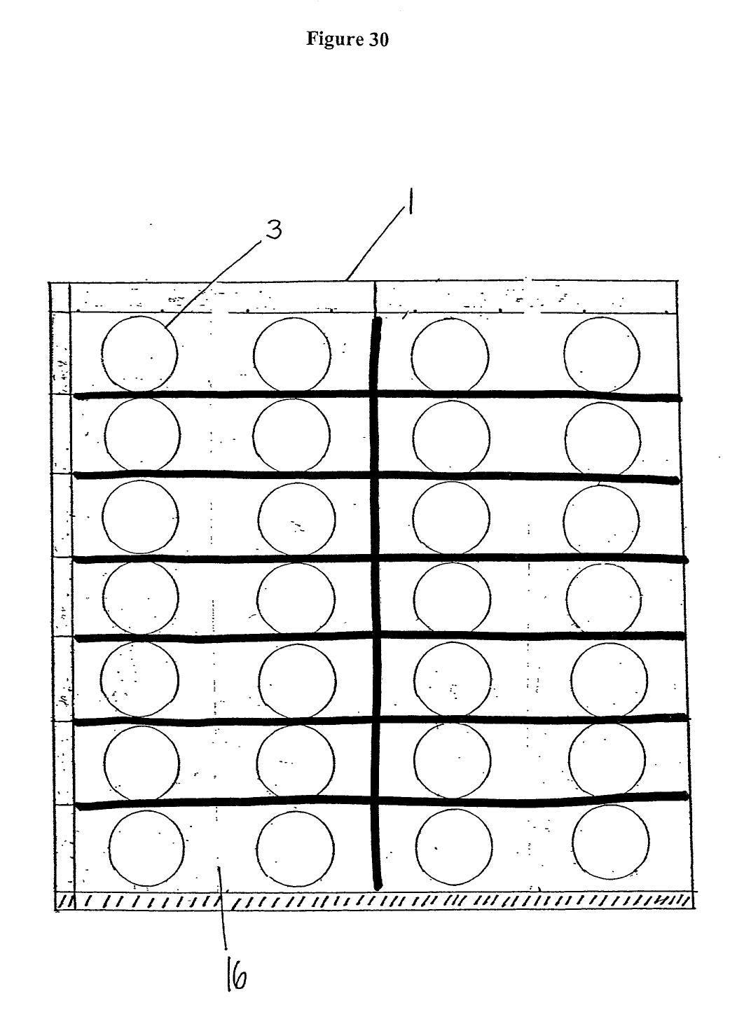

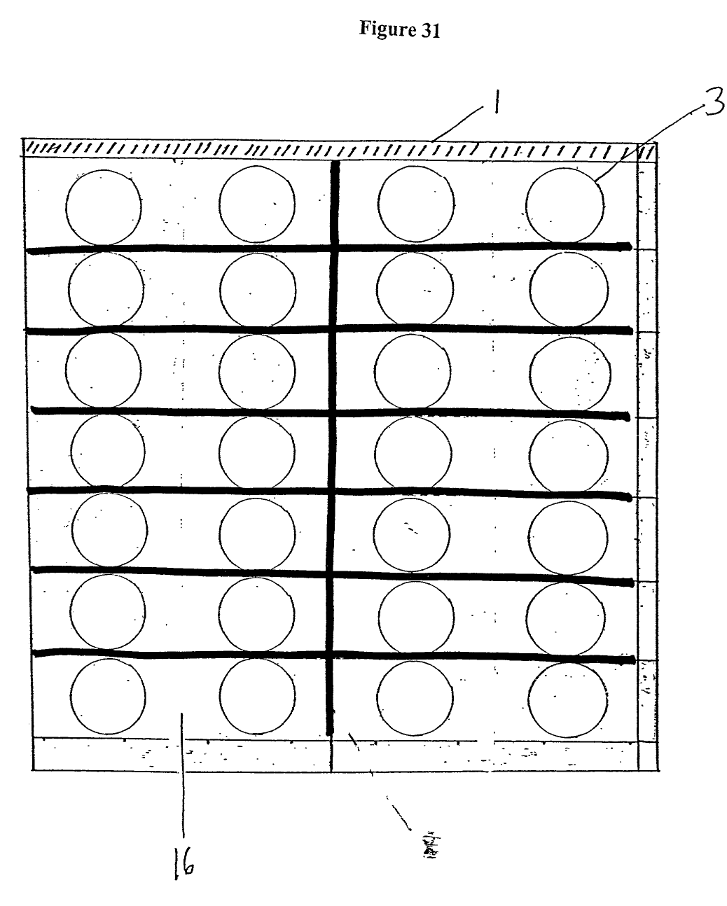

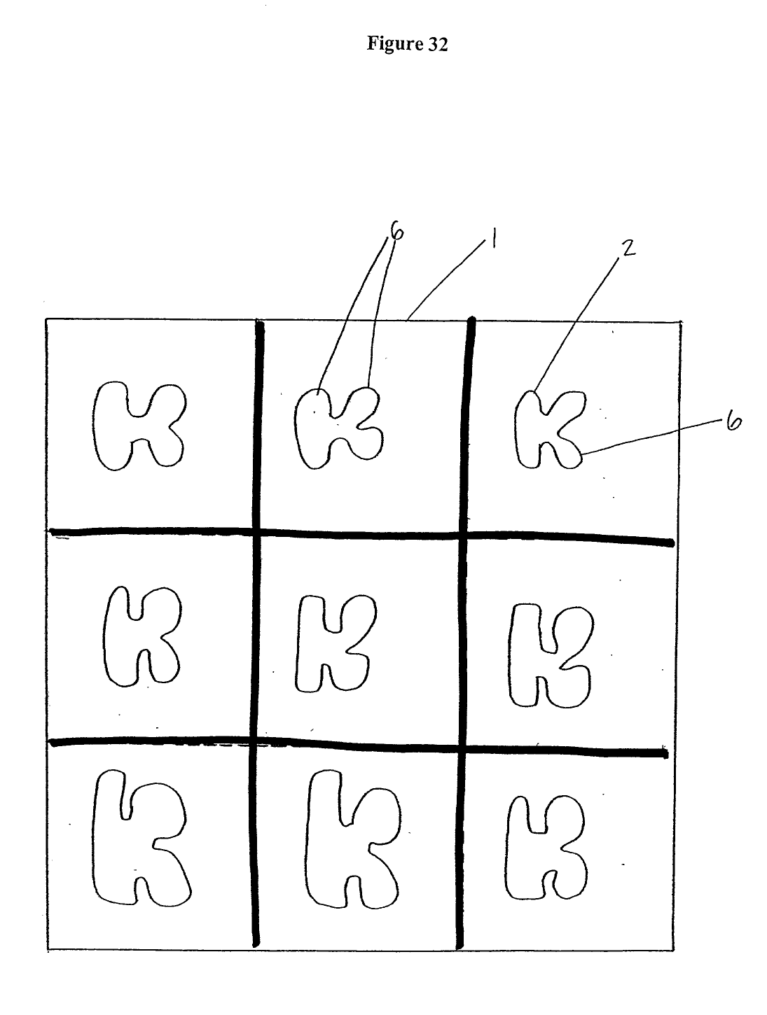

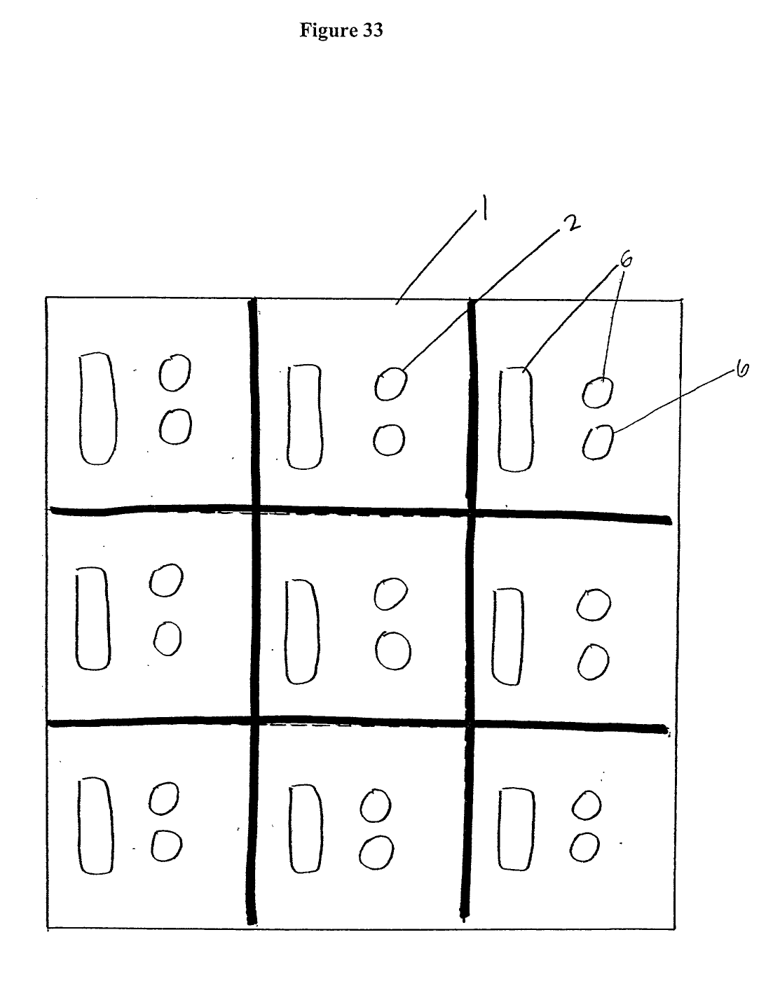

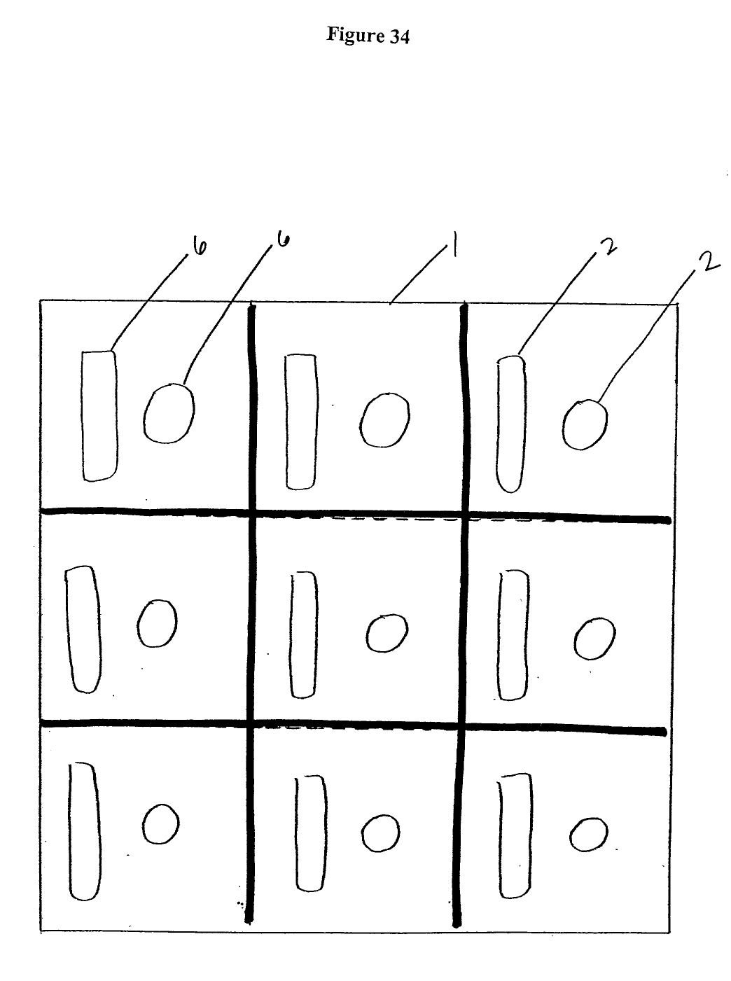

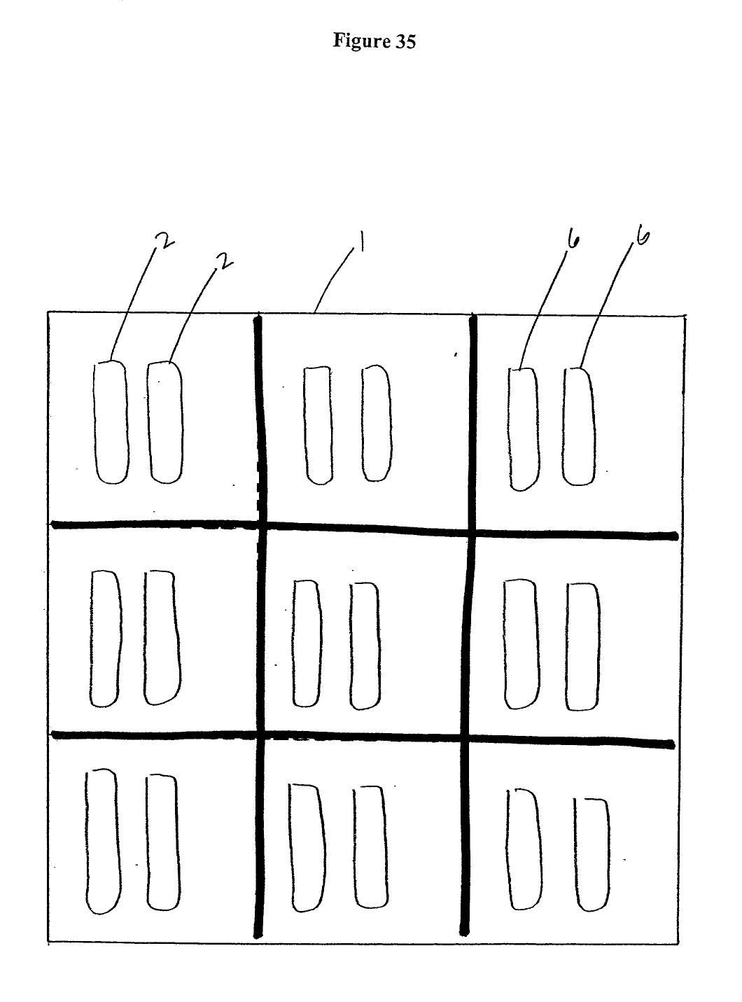

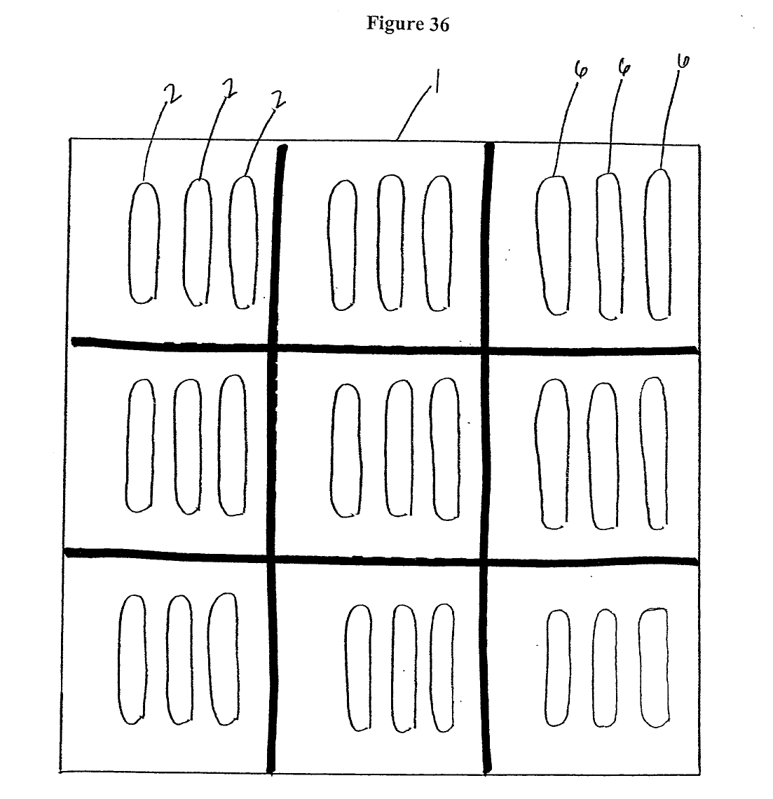

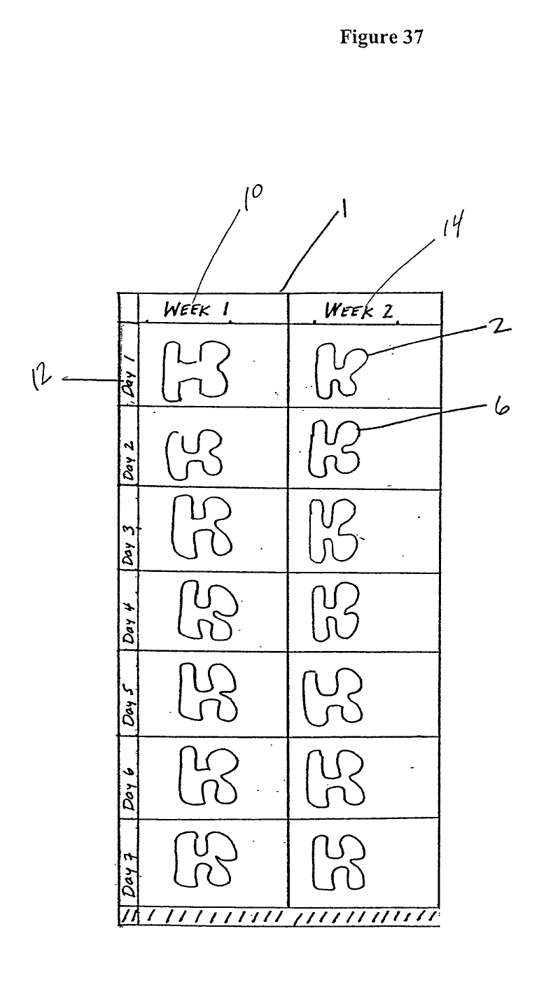

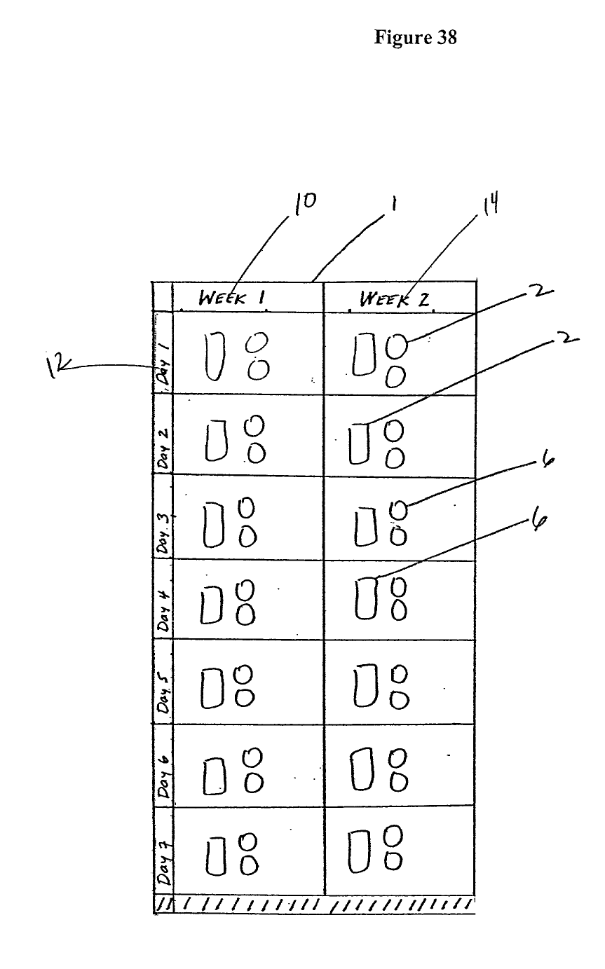

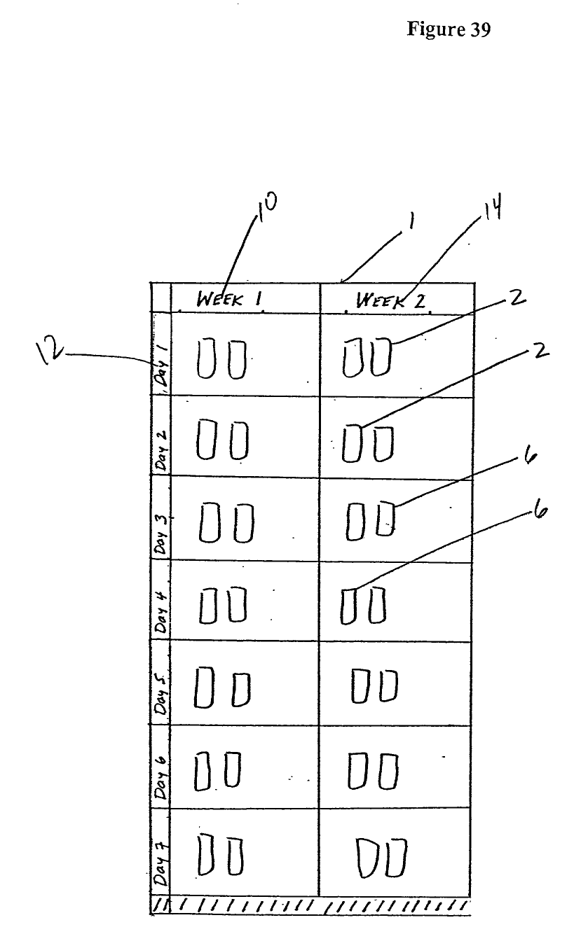

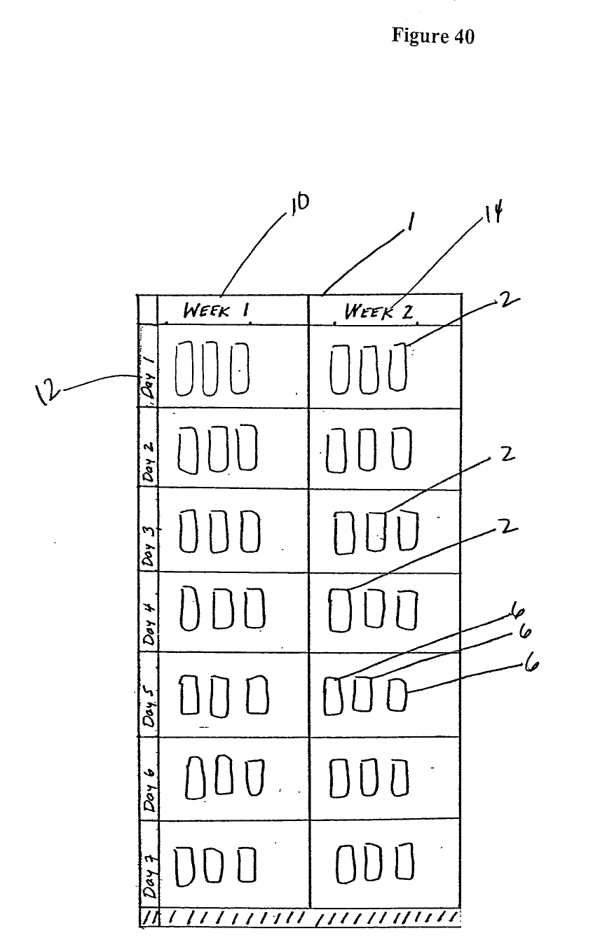

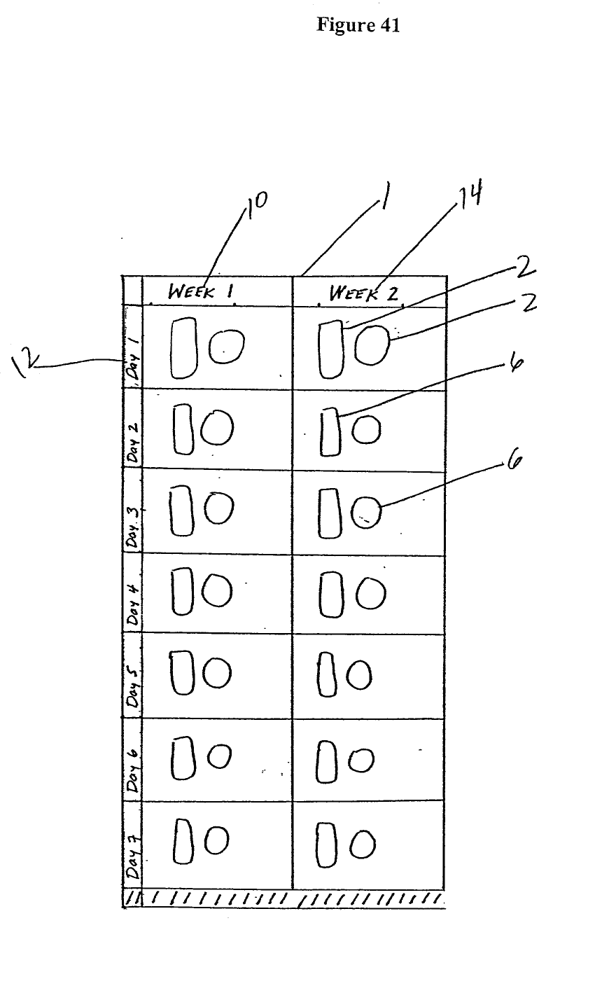

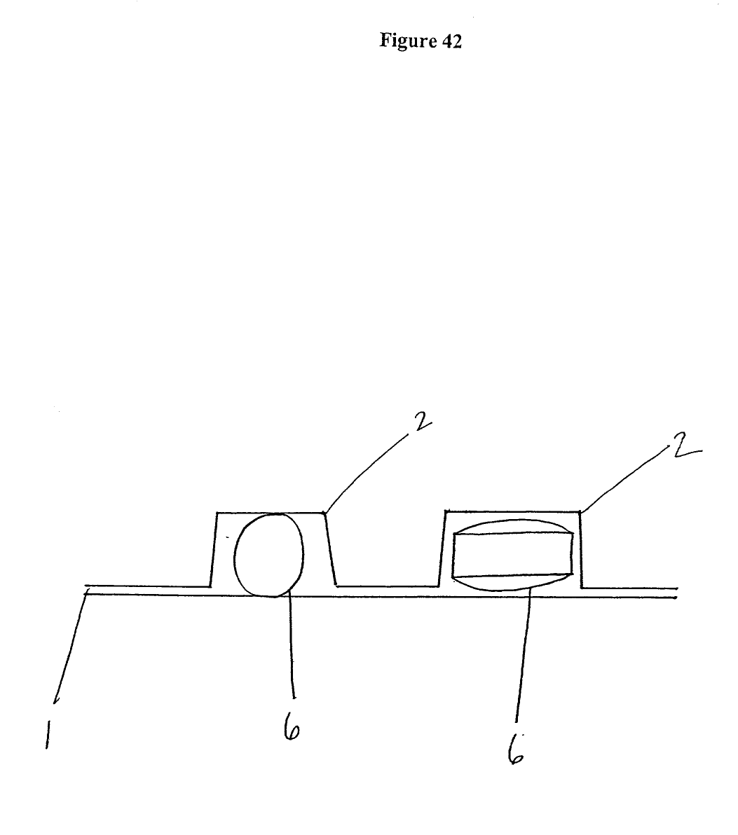

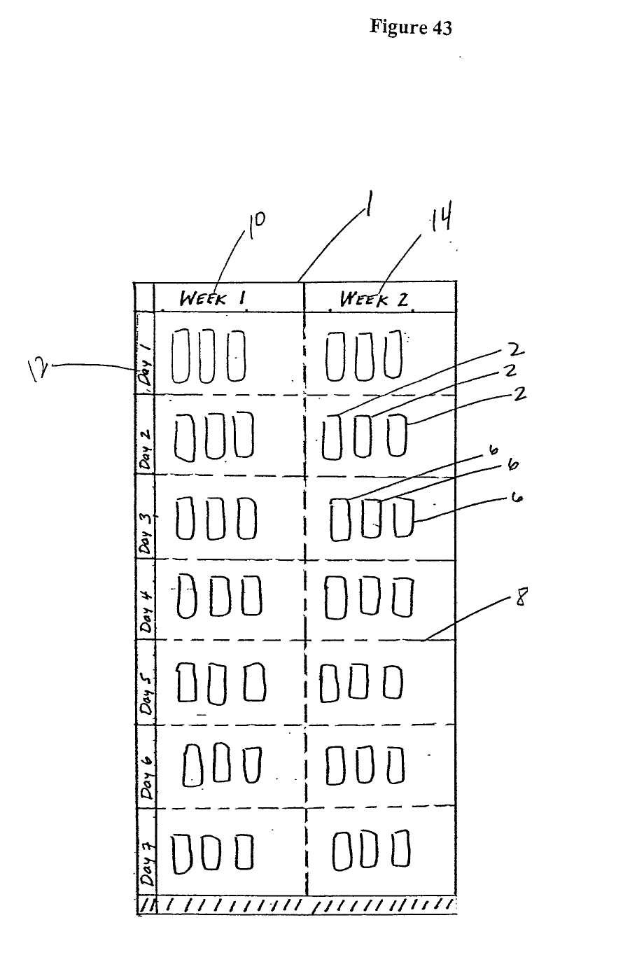

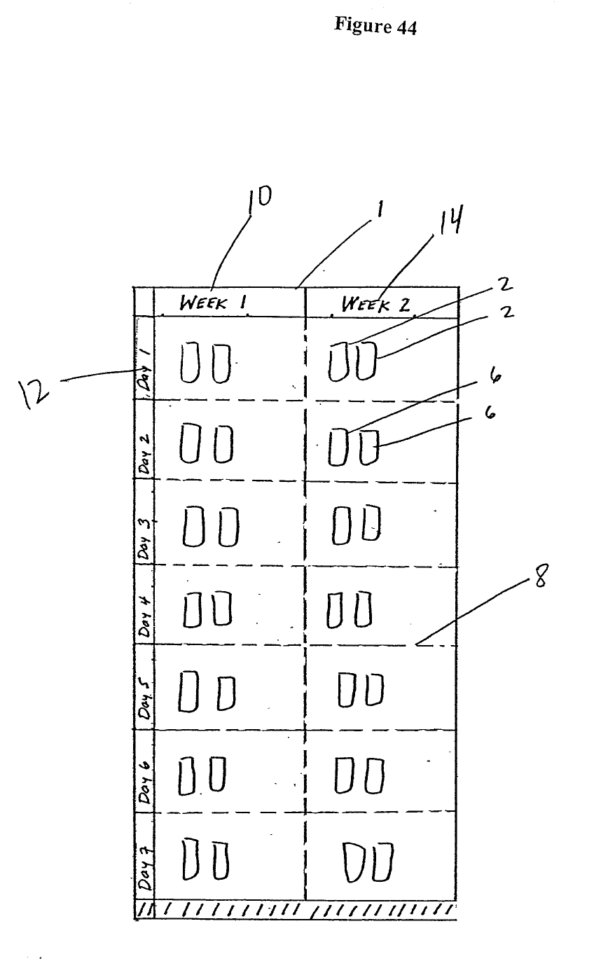

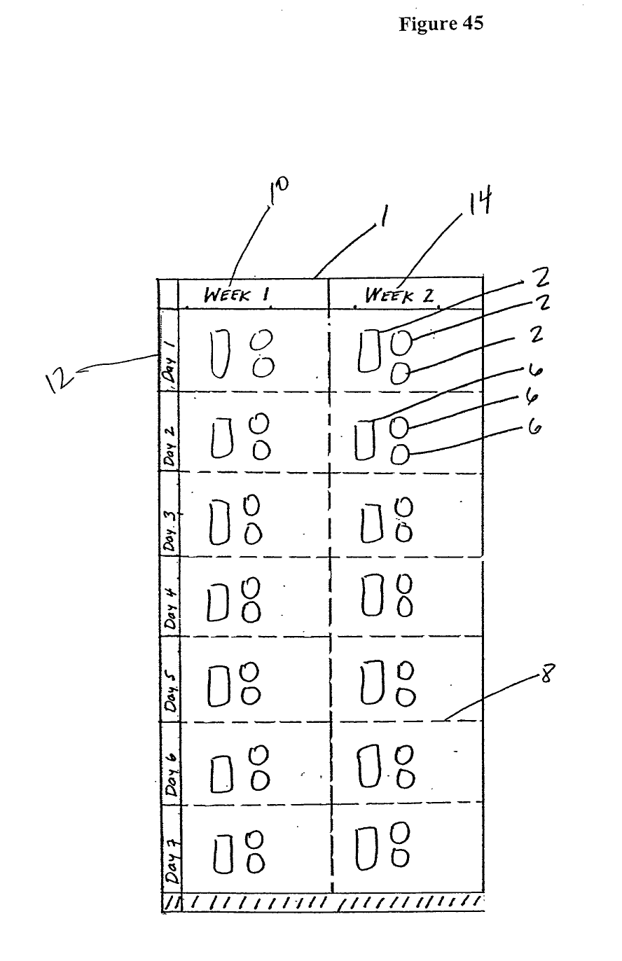

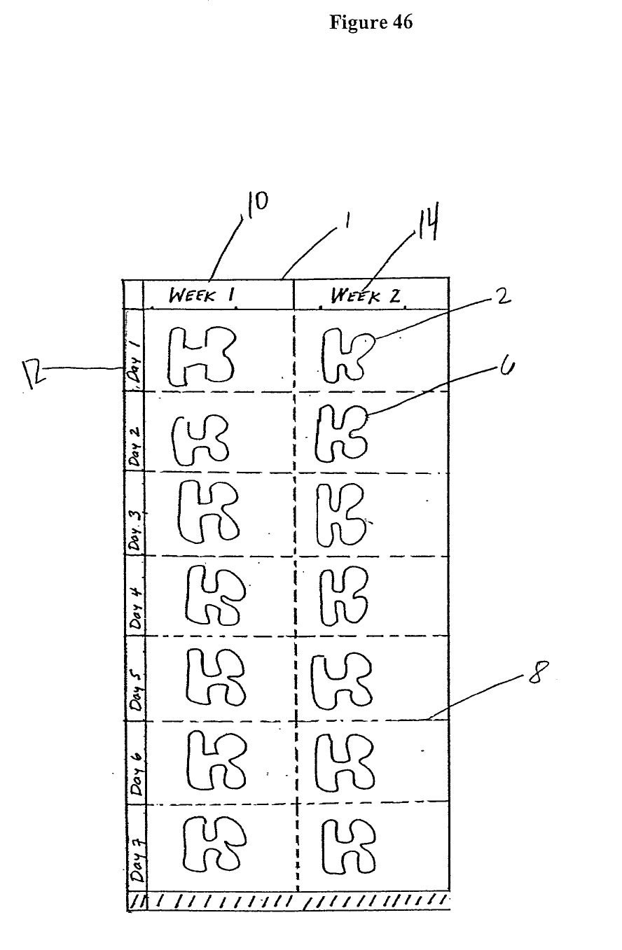

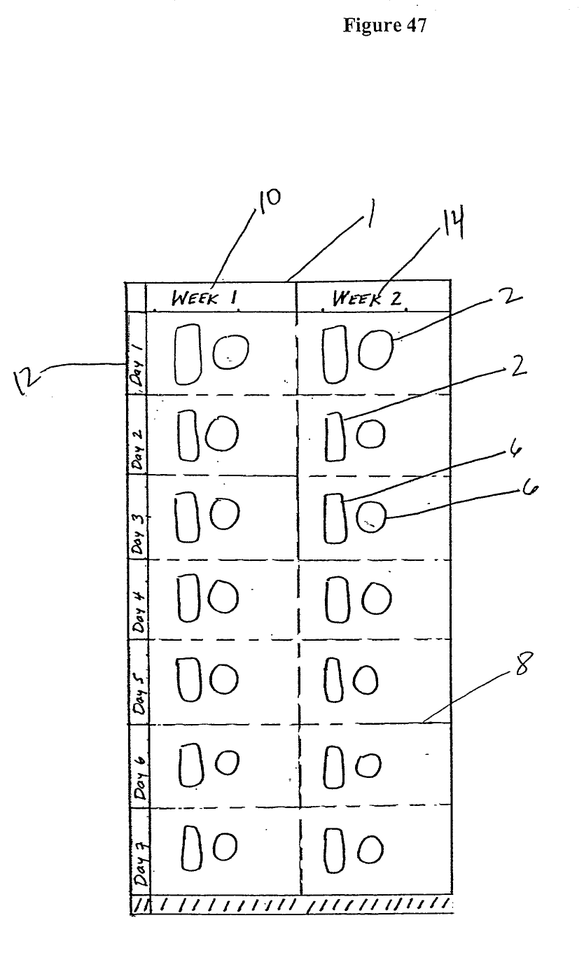

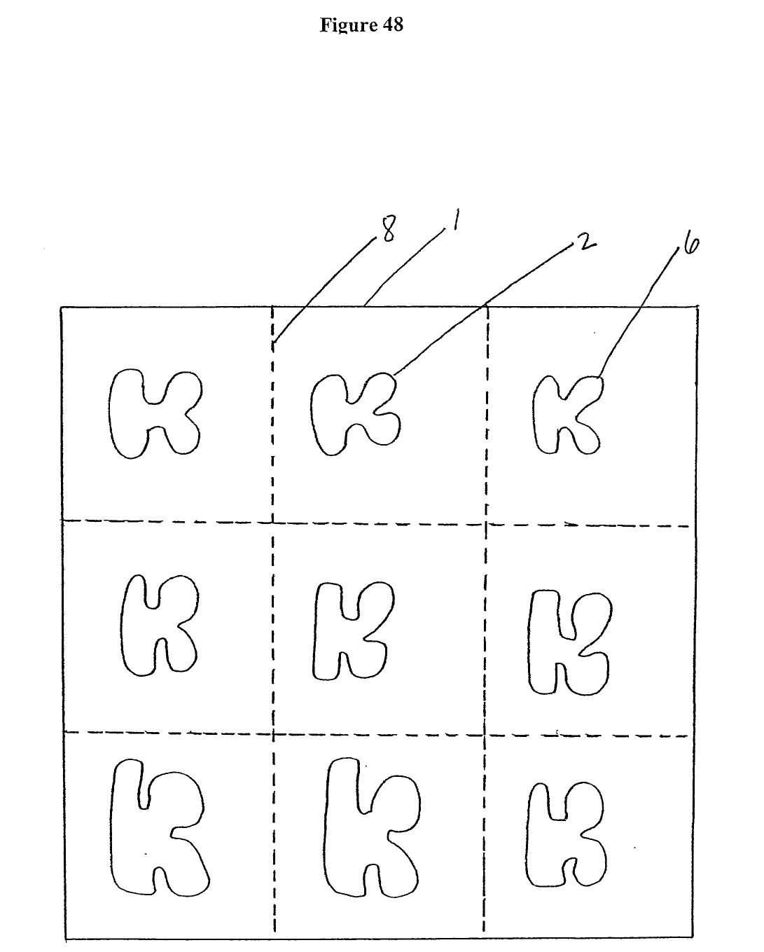

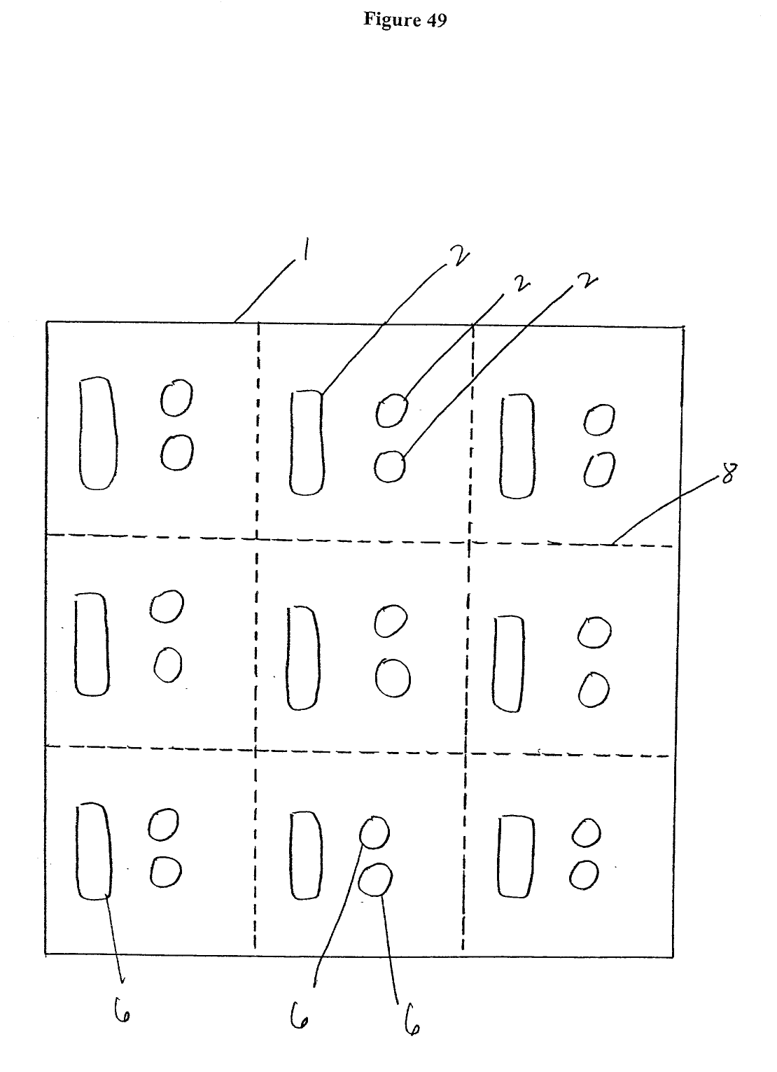

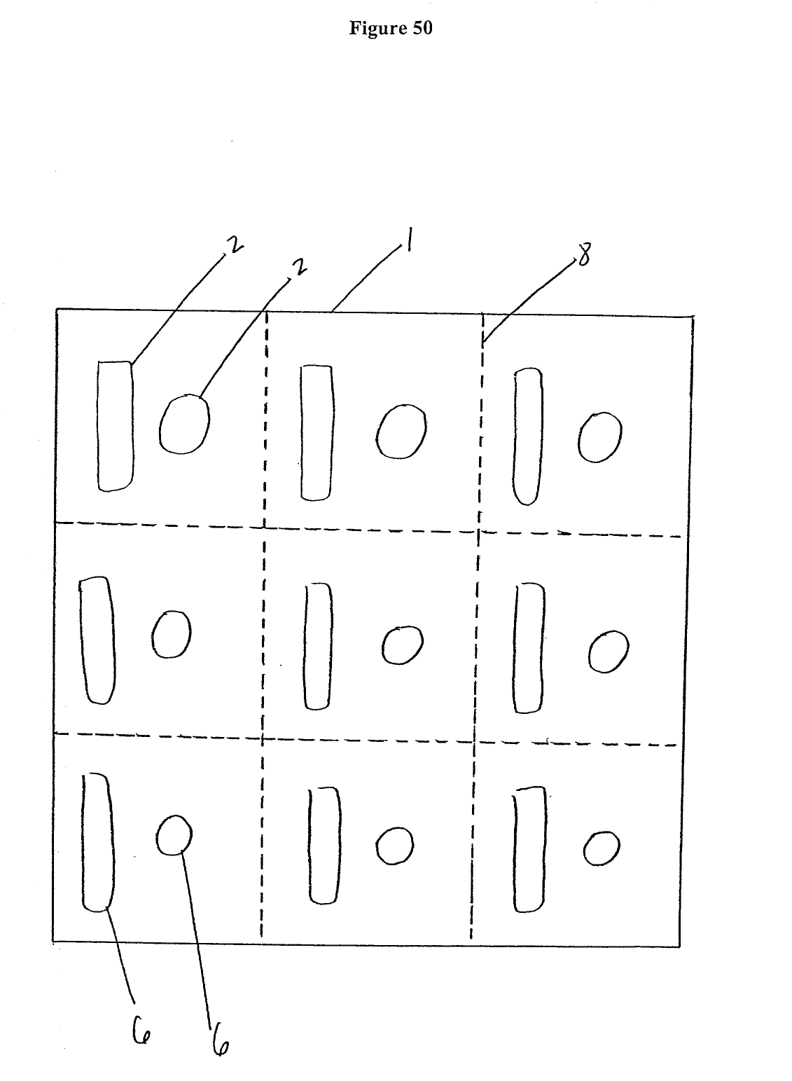

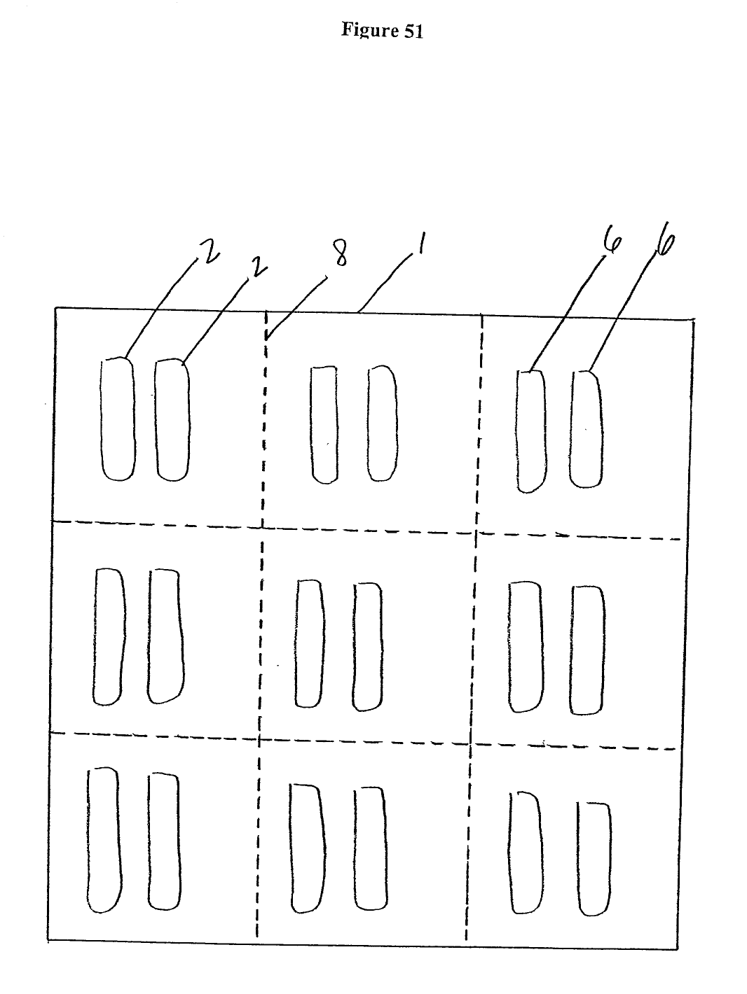

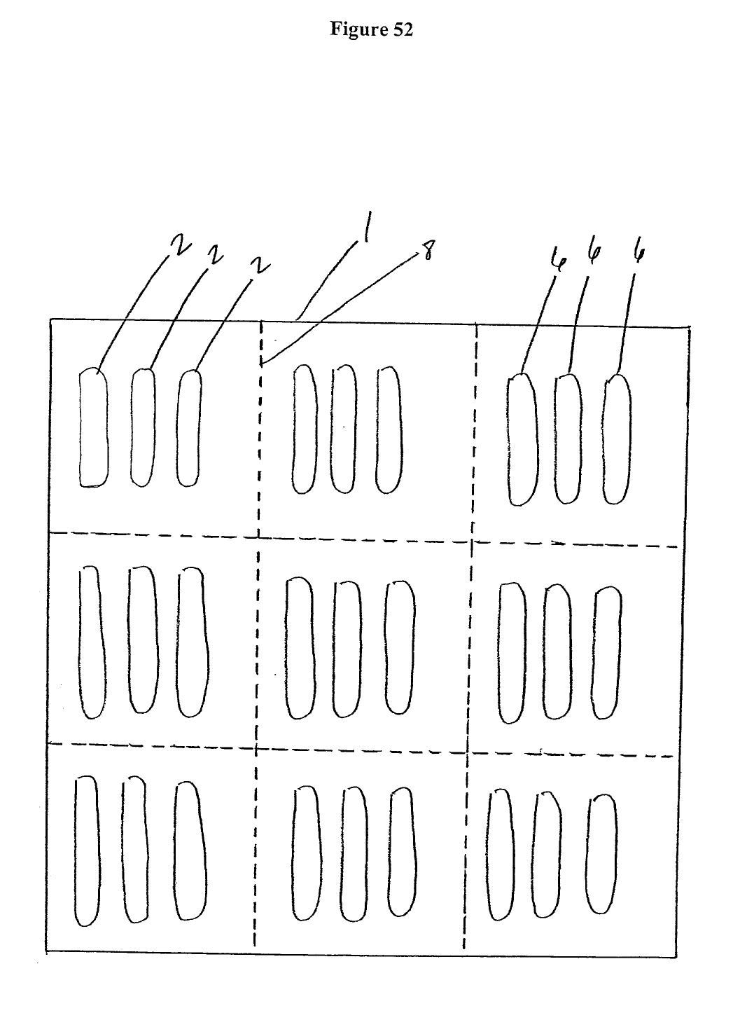

The blister package 1 can preferably include a backing 4 (see, e.g., FIG. 25). If present, the backing 4 can have any suitable shape, provided the rupturable substrate 3 and each of the one or more blister layers 2 can be contained on the backing 4. In one embodiment, the backing 4 can have a rectangular shape. In another embodiment, the backing 4 can have a circular shape.

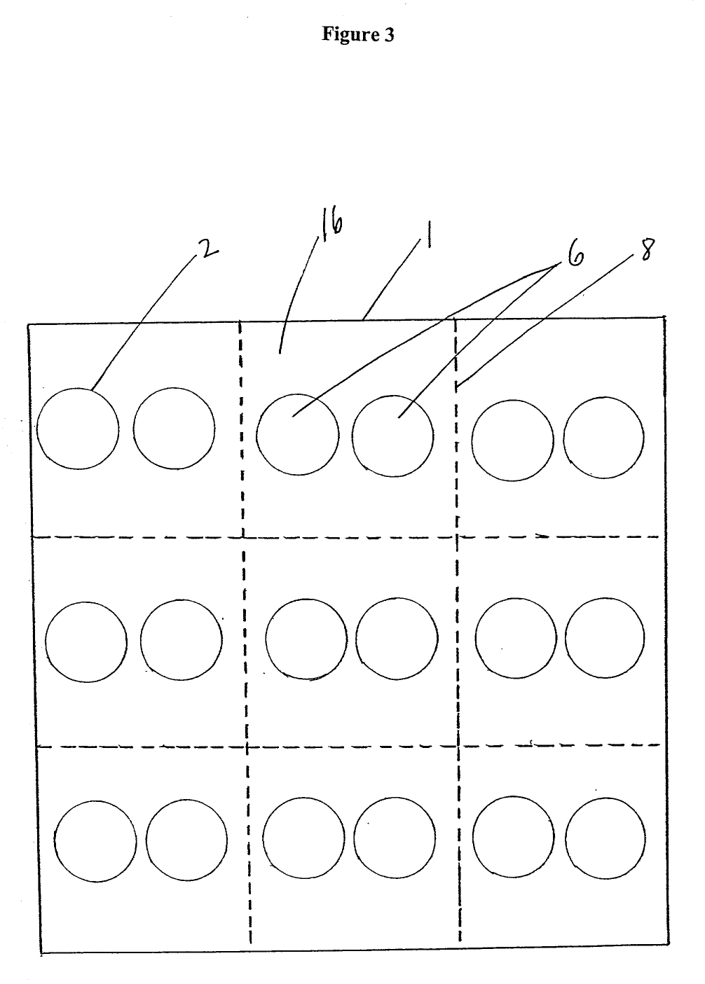

The blister package 1 can include perforations 8 such that one or two blister layers 2 can exist on a single piece 16 of blister package 1, as shown in FIGS. 1-8. In one embodiment, the package 1 can include perforations 8 such that one blister layer 2 can exist on a single piece 16 of blister package 1, as shown in FIGS. 1-2, 5-6, and 23-24. In such an embodiment, each of the blister layers 2 can include both the one or more proton pump inhibitors and the one or more NSAIDS. Alternatively, the blister package 1 can include perforations 8 such that two blister layers 2 can exist on a single piece 16 of blister package 1, as shown in FIGS. 3-4, 7-8, and 21-22. In such an embodiment, each of the blister layers 2 can include either the unit dosage form of the proton pump inhibitor or the unit dosage form of the NSAID. The perforations 8 allow a patient to easily remove a discrete dosage (e.g., daily dosage) of medicament 6.

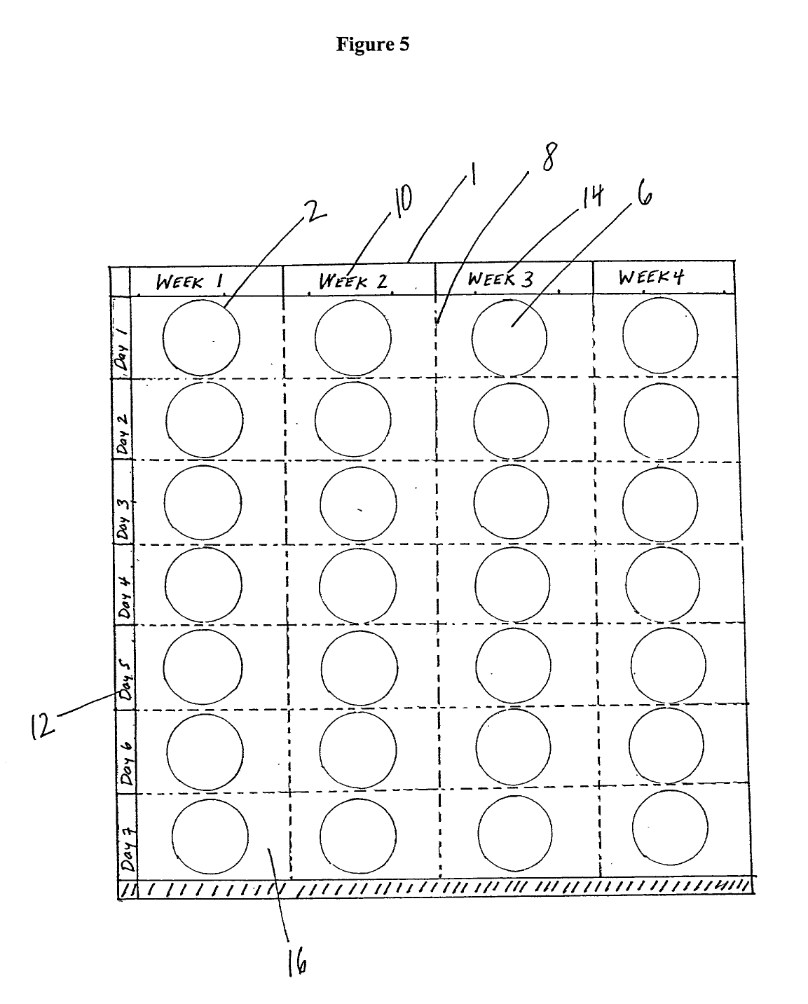

The blister package 1 can optionally include indicia 10 printed on the rupturable substrate 3 indicating the sequence of removal of the proton pump inhibitor and the non-steroidal anti-inflammatory drug from each of the of the blister layers 2 (see, e.g., FIGS. 5, 7, 13, and 15). In one embodiment, the indicia 10 is printed on the rupturable substrate 3. In another embodiment, the indicia 10 is printed on the backing 4. In such an embodiment, the indicia 10 is printed on a relevant location (e.g., proximally close to the blister layer 2) to aid the patient in dispensing the medicament 6. Specifically, the indicia 10 can illustrate the day 12 and/or the week 14 when the medicament 6 in a specific blister layer 2 is to be administered.

The medicament 6 is a unit dosage form of one or more proton pump inhibitors, a unit dosage form of one or more nonsteroidal anti-inflammatory drugs, or a combination thereof.

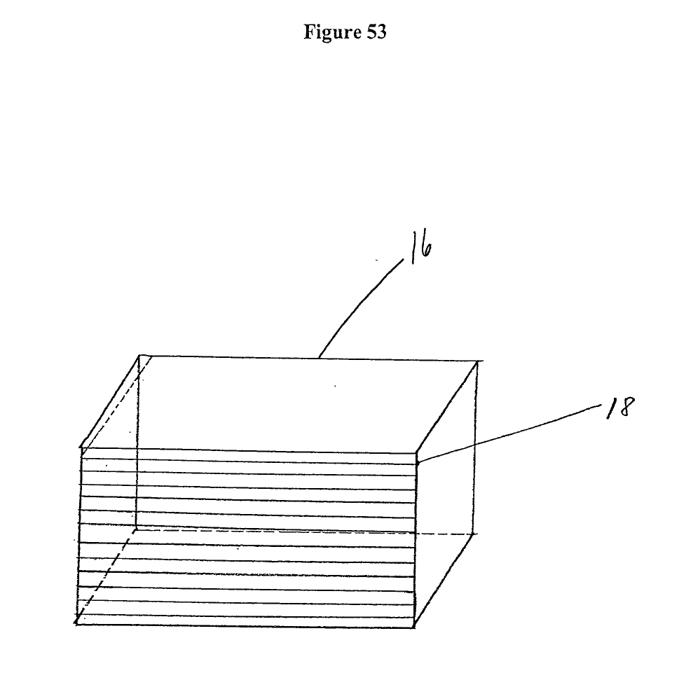

As illustrated in FIG. 53, the invention also provides a blister pack rack 17 having a plurality of shelves 18, suitable for holding, storing, shipping, or dispensing a plurality (e.g. 1, 2, 3, 4, 5, 10, 15, or 20) of blister cards or drug packaging systems of the invention.

Proton Pump Inhibitor

As used herein, a “proton pump inhibitor” is an antisecretory compound, that suppresses gastric acid secretion by inhibition of the H⁺/JK⁺ ATPase enzyme system. Suitable proton pump inhibitors for use in the combination packages of the instant invention are disclosed, e.g., in Physician's Desk Reference (PDR), Medical Economics Company (Montvale, N.J.), (53rd Ed.), 1999; Mayo Medical Center Formulary. Unabridged Version, Mayo Clinic (Rochester, Minn.), January 1998; and Merck Index, An Encyclopedia of Chemicals, Drugs, and Biologicals, (11th Ed.), Merck & Co., Inc. (Rahway, N.J.), 1989.

One suitable proton pump inhibitor is omeprazole, which is 5methoxy-2-[[(4-methoxy-3, 5-dimethyl-2-pyridinyl)methyl]sulfinyl]- 1H-benzimidazole. Omeprazole is commercially available from Astra Pharmaceuticals as Prilosec® (omeprazole). Another suitable proton pump inhibitor is lansoprazole, which is 2-[[[8-methyl-4-(2,2,2-trifluoroethoxy)-2-pyridyl]methyl]sulfinyl]benzimidazole. Lansoprazole is commercially available from Tap Pharmaceuticals as Prevacid® (lansoprazole). Another suitable proton pump inhibitor is esomeprazole.

Proton Pump Inhibitor Dosages

Any suitable amount of proton pump inhibitor can be employed, provided the amount of proton pump inhibitor administered effectively suppresses gastric acid secretion by specific inhibition of the H⁺/K⁺ ATPase enzyme system at the secretory surface of the gastric parietal cell. Preferably, the dosage of proton pump inhibitor will correspond to a dosage that is approved for administration by a governmental regulatory authority (e.g. the U.S. FDA). For example, suitable doses for proton pump inhibitors are disclosed in the Physician's Desk Reference (PDR), Medical Economics Company (Montvale, N.J.), (53rd Ed.), 1999. Typically, the amount of proton pump inhibitor will depend on the specific proton pump inhibitor employed.

For example, the recommended adult dosage of omeprazole for the short-term treatment of active duodenal ulcer is 20 mg once daily (orally), for four to eight weeks. The recommended adult dosage of omeprazole for gastric ulcer is 40 mg once daily (orally), for four to eight weeks. The recommended adult dosage of omeprazole for symptomatic gastroesophageal reflux disease (GERD) with no esophageal lesions is 20 mg daily for up to 4 weeks. The recommended adult oral dose for the treatment of patients with erosive esophagitis and accompanying symptoms due to GERD is 20 mg daily for 4 to 8 weeks. The recommended adult dosage of omeprazole for maintenance of healing of erosive esophagitis is 20 mg once daily (orally). The recommended adult dosage of omeprazole for pathological hypersecretory conditions varies with the individual patient. For example, the recommended adult oral starting dose is 60 mg omeprazole once a day. Doses up to 120 mg t.i.d. have been administered. Typically daily dosages of greater than 80 mg should be administered in divided doses.

The recommended adult dosage of lansoprazole for the short-term treatment of duodenal ulcer is 15 mg once daily (orally) for 4 weeks. The recommended adult dosage of lansoprazole for the maintenance of healed duodenal ulcers is 15 mg once daily (orally). The recommended adult dosage of lansoprazole for the short-term treatment of gastric ulcer is 30 mg once daily (orally) for up to eight weeks. The recommended adult dosage of lansoprazole for the short-term treatment of symptomatic gastroesophageal reflux disease (GERD) is 15 mg once daily (orally) for up to 8 weeks. The recommended adult dosage of lansoprazole for the short-term treatment of erosive esophagitis is 30 mg once daily (orally) for up to 8 weeks. For patients who do not heal with lansoprazole for 8 weeks (5-10%), it may be helpful to give an additional 8 weeks of treatment. If there is a recurrence of erosive esophagitis, an additional 8-week course of lansoprazole may be considered. The recommended adult dosage of lansoprazole for the healing of erosive esophagitis is 15 mg once daily (orally).

Preferably, for use in the packaging system of the invention, the proton pump inhibitor is omeprazole. More preferably, the proton pump inhibitor is omeprazole, present as a 20 mg tablet or capsule (see for example U.S. Pat. No. 6,077,541).

Nonsteroidal Anti-Inflammatory Drug

The term “nonsteroidal anti-inflammatory drug” includes any analgesic agent that does not include a steroidal ring structure, framework, or backbone. Suitable nonsteroidal anti-inflammatory drugs for use in the packaging system of the invention are disclosed, e.g., in Physician's Desk Reference (PDR), Medical Economics Company (Montvale, N.J.), (53rd Ed.), 1999; Mayo Medical Center Formulary. Unabridged Version, Mayo Clinic (Rochester, Minn.), January 1998; and Merck Index, An Encyclopedia of Chemicals, Drugs, and Biologicals, (11th Ed.), Merck & Co., Inc. (Rahway, N.J.), 1989.

Suitable nonsteroidal anti-inflammatory drugs include, naproxen, diclofenac, sulindac, oxaprozin, diflunisal, aspirin, piroxicam, indomethacin, etodolac, ibuprofin, fenoprofen, ketoprofen, mefenamic acid, nabumetone, tolmetin, and ketorolac, and pharmaceutically acceptable salts thereof

Anaprox® (naproxen), Naprosyn® (naproxen), and EC-Naprosyn® (naproxen) are commercially available from Roche Laboratories. The active ingredient is (S)-6-methoxy-α-methyl-2-naphthaleneacetic acid, sodium salt.

Voltaren® (diclofenac sodium) and Voltaren® XR (once daily diclofenac sodium) is commercially available from Novartis and generic diclofenac sodium is commercially available from Novapharm, Geneva, and Roxane. The active ingredient is 2-[2,6-dichlorophenyl)amino]-benzeneacetic acid, monosodium salt.

Cataflam® (diclofenac potassium) is commercially available from Novartis. The active ingredient is 2-[(2-,6-dichlorophenyl)amino] benzeneacetic acid, monopotassium salt.

Clinoril® (sulindac) is commercially available from Merck. The active ingredient is (Z)-5-fluoro-2-methyl-1-[[p-(methylsulfinyl)phenyl]-methylene]-1H-indene-3-acetic acid.

Daypro® (oxaprozin) is commercially available from Searle. The active ingredient is 4,5-diphenyl-2-oxazole-propionic acid.

Dolobid® (diflunisal) is commercially available from Merck. The active ingredient is 2′,4′-difluoro-4-hydroxy-3-biphenylcarboxylic acid.

Ecotrin® (enteric coated aspirin) is commercially available from SmithKline Beecham Consumer. The active ingredient is acetylsalicylic acid, ASA.

Feldene® (piroxicam) is commercially available from Pfizer. The active ingredient is 4-hydroxy-2-methyl-N-2-pyridinyl-2H-1,2-benzothiazine-3-carboxamide 1,1-dioxide, an oxicam.

Indocin® (indomethacin) is commercially available from Merck. The active ingredient is 1-(4-chlorobenzoyl)-5-methoxy-2-methyl-1H-indole-3-acetic acid.

Lodine® (etodolac) and Lodine® ER (once daily etodolac) is commercially available from Wyeth-Ayerst. The active ingredient is (±) 1,8-diethyl-1,3,4,9-tetrahydropyrano-[3,4-b]indole-1-acetic acid.

Motrin® (ibuprofin) is commercially available from McNeil Consumer. The active ingredient is (±)-2-(p-isobutylphenyl) propionic acid.

Nalfon® (fenoprofen) is commercially available from Dista. The active ingredient is α-methyl-3-phenoxy, calcium salt dihydrate.

Naprelan® (naproxen sodium) is commercially available from Wyeth-Ayerst. The active ingredient is 6-methoxy-α-methyl-2-naphthaleneacetic acid sodium salt.

Orudis® (ketoprofen) and Oruvail® (ketoprofen) are commercially available from Wyeth-Ayerst. The active ingredient is 2-(3-benzoylphenyl)-propionic acid.

Ponstel® (mefenamic acid) is commercially available from Parke-Davis. The active ingredient is N-(2,3-xylyl)-anthranilic acid.

Relafen® (nabumetone) is commercially available from SmithKline Beecham. The active ingredient is 4-(6-methoxy-2-naphthalenyl)-2-butanone.

Tolectin® (tolmetin sodium) is commercially available from Ortho-McNeil Pharmaceutical. The active ingredient is sodium 1-methyl-5-(4-methylbenzoyl)-1H-pyrrole-2-acetate dihydrate.

Toradol® (ketorolac) is commercially available from Roche Laboratories. The active ingredient is (±)-5-benzoyl-2,3-dihydro-11H-pyrrolizine-1-carboxylic acid, compound with 2-amino-2-(hydroxymethyl)-1,3-propanediol.

Dosages of Nonsteroidal Anti-Inflammatory Drug

Any suitable amount of nonsteroidal anti-inflammatory drug can be employed in the packaging system of the invention. Preferably, the dosage of NSAID will correspond to a dosage that is approved for administration by a governmental regulatory authority (e.g. the U.S. FDA), for example, as disclosed in Physician's Desk Reference (PDR), Medical Economics Co., 53rd Ed., 1999. Typically, the amount of NSAID will depend on the specific NSAID employed.

For naproxen the recommended starting dose can be 550 mg followed by 550 mg every 12 hours or 275 mg every 6 to 8 hours, as required. The recommended dose could also be 750 to 1000 mg once daily. Alternatively, the recommended dose can be 1000 to 1500 mg once daily. Alternatively, the recommended dose can be 1000 to 1500 mg on the first day, followed by 1000 mg once daily until the symptoms have subsided.

For diclofenac the recommended dose can be 100 to 150 mg per day b.i.d or t.i.d. Alternatively, the recommended dose can be 150 mg per day. Alternatively, the recommended dose can be 50 mg followed by doses of 50 mg every 8 hours. Alternatively, the recommended daily dose can be 25 mg, 50 mg, or 75 mg once a day.

For sulindac the recommended starting dose can be 150 mg twice a day or 200 mg twice a day.

For oxaprozin the recommended daily dose can be 1200 mg. Alternatively, the recommended daily dose can be 600 to 1200 mg depending on severity of the disease and body size of patient.

For diflunisal the recommended dose is an initial dose of 500 to 1000 mg followed by 250 to 500 mg every 8 to 12 hours depending on severity of pain and body size of patient. Alternatively, the recommended dose can be 500 to 1000 mg daily in divided doses.

For enteric the recommended dose can be 300 to 325 mg daily. Alternatively, the recommended dose can be 160 to 162.5 mg daily. Alternatively, the maximum dosage can be 4000 mg per day in divided doses of up to 650 mg every 4 hours.

For piroxicam the recommended dose can be a single daily dose of 20 mg.

For indomethacin the recommended dose can be an initial dose of 25 mg b.i.d. or t.i.d. followed by 25 to 50 mg if required by continuing symptoms and depending on patient tolerance. Alternatively, the recommended dose can be 100 to 200 mg daily.

For etodolac the recommended dose can be up to 1200 mg daily, given as 200 to 400 mg every 6 to 8 hours. Alternatively, the recommended dose can be 300 mg b.i.d., t.i.d. or 400 to 500 b.i.d. For long-term treatment, the recommended dose can be 600 to 1200 mg per day, depending on severity of disease and patient tolerance.

For ibuprofin the recommended dose can be 3-6 50 mg tablets every 6 to 8 hours as needed; 2-4 160 mg caplets or tablets every 6 to 8 hours as needed; 1-2 200 mg tablets, caplets, or gelcaps every 4 to 6 hours; or 1-2 tablets or caplets every 4 to 6 hours.

For fenoprofen the recommended dose can be 200 mg every 4 to 6 hours as needed. Alternatively, the recommended dose can be 300 to 600 mg 3 or 4 times per day depending on the severity of the symptoms and the tolerance of the patient.

For ketoprofen the recommended dose can be 75 mg three times daily or 50 mg four times daily, or 200 mg once daily.

For mefenamic acid the recommended dose can be 500 mg initially, followed by 250 mg every six hours as needed.

For nabumetone the recommended dose can be a starting dose of 1000 mg, followed by a daily dose of 1000 to 2000 mg depending on patient need and tolerance.

For tolmetin sodium the recommended starting dosage for children age 2 and older can be 15-30 mg/kg/day, preferably, 20 mg/kg day. The recommended starting dose for treatment of adults can be 600-1800 mg daily, preferably 400 mg three times daily (1200 mg daily).

In one embodiment, the NSAID can be diclofenac sodium, naproxen sodium, naproxen, or nabumetone, or a combination thereof.

In another embodiment, the NSAID is preferably diclofenac sodium. More preferably, the NSAID is diclofenac sodium, present as a 25 mg tablet, a 50 mg tablet, or a 75 mg tablet.

In another embodiment, the NSAID is preferably nabumetone. More preferably, the NSAID is nabumetone present as a 500 mg tablet or a 750 mg tablet.

In another embodiment, the NSAID is a combination of naproxen sodium and naproxen. The naproxen sodium and naproxen, can exist as two separate tablets or can exist in a single tablet. Preferably, the naproxen sodium and naproxen, are provided as two separate tablets. More preferably, the naproxen sodium is present as a single 1000 mg tablet or as two 500 mg tablets and the naproxen is present as a single tablet of 250 mg or 750 mg.

In one preferred embodiment of the present invention, two 500 mg tablets of naproxen sodium, or naproxen, and one 20 mg tablet of omeprazole are packaged together for daily co-administration to humans. In another preferred embodiment of the present invention, one 500 mg or 750 mg tablet of nabumetone and one 20 mg tablet of omeprazole are packaged together for daily co-administration to humans. In another embodiment of the present invention, one 25 mg tablet, 50 mg tablet, or 75 mg tablet of diclofenac sodium and one 20 mg tablet of omeprazole are packaged together for daily co-administration to humans.

All publications, patents, and patent documents are incorporated by reference herein, as though individually incorporated by reference.

What is claimed is:

1. A drug packaging system comprising packaging material comprising therein combined prescription drug therapy comprising one or more unit dosage forms of a first drug and one or more unit dosage forms of a second drug, wherein said first and second drug are independently selected from the group consisting of non-steroidal anti-inflammatory drugs, proton pump inhibitors, calcium channel blockers, angiotensin converting enzyme (ACE) inhibitors, anti-depressants, selective serotonin reuptake inhibitors, antihistamines, decongestants, biguanides, sulfonylureas, 3-hydroxy-3-methylglutaryl coenzyme A (HMG CoA) reductase inhibitors, anti-epileptic, and anti diabetics.

2. A drug packaging system comprising packaging material comprising therein combined prescription drug therapy comprising one or more unit dosage forms of a first drug and one or more unit dosage forms of a second drug, wherein at least one of said first or second drug are selected from the group consisting of non-steroidal anti-inflammatory drugs, calcium channel blockers, angiotensin converting enzyme (ACE) inhibitors, anti-depressants, selective serotonin reuptake inhibitors, antihistamines, decongestants, biguanides, sulfonylureas, 3-hydroxy-3-methylglutaryl coenzyme A (HMG CoA) reductase inhibitors, anti-epileptic, and anti diabetics.

3. A drug packaging system comprising packaging material comprising therein combined prescription drug therapy comprising one or more unit dosage forms of a first drug and one or more unit dosage forms of a second drug, wherein said first and second drug are independently selected from the group consisting of antibiotics and anti-ulcer agents selected from the group consisting of H2 antagonists, antacids, bismuth compounds, prostaglandins, carbenoxolone and anticholinergic agents.

4. The drug packaging system of claim 1 wherein said first comprises a nonsteroidal anti-inflammatory drug and said second drug comprises a proton pump inhibitor.

5. The drug packaging system of claim 4 wherein said proton pump inhibitor is omeprazole, lansoprazole, esomeprazole, pantoprazole, isomers, enantiomers or pharmaceutically acceptable salts thereof.

6. The drug packaging system of claim 1 wherein each unit dosage form is independently selected from the group consisting of a tablet, capsule, gel cap, and a caplet.

7. The drug packaging system of claim 4 wherein said non-steroidal anti-inflammatory drug is naproxen, diclofenac, sulindac, oxaprozin, diflunisal, aspirin, piroxicam, indomethacin, etodolac, ibuprofen, fenoprofen, ketoprofen, mefenamic acid, nabumetone, tolmetin, ketorolac, or any pharmaceutically acceptable salt thereof.

8. The drug packaging system of claim 4 wherein said non-steroidal anti-inflammatory drug is diclofenac or a pharmaceutically acceptable salt thereof.

9. The drug packaging system of claim 4 wherein said proton pump inhibitor is omeprazole.

10. The drug packaging system of claim 4 wherein each said unit dosage form is a daily dose for a human patient.

11. The drug packaging system of claim 1 wherein said system is configured as a blister package comprising:
a) a rupturable substrate
b) a layer forming one or more blisters over the rupturable substrate; wherein each of the one or more blisters contain one or more unit dosage forms.

12. The drug packaging system of claim 11 wherein said first drug is a non-steroidal anti-inflammatory drug and said second drug is a proton pump inhibitor.

13. The drug packaging system of claim 11 wherein said proton pump inhibitor is omeprazole.

14. The drug packaging system of claim 11 wherein said non-steroidal anti-inflamatory drug is diclofenac.

15. The drug packaging system of claim 1 wherein said system comprises a plurality of blister packs contained within a single package.

16. The drug packaging system of claim 1 wherein said system comprises unit doses for up to 28 days.

17. The drug packaging system of claim 1 wherein said system comprises unit doses for 7-14 days.

18. The drug packaging system of claim 1 wherein said system comprising one or more dosage forms of a third drug.

19. A method of treating a disease or condition treatable by combined administration of more than one medicament, said method comprising:
providing a drug packaging system of comprising one or more unit dosage forms of a first drug and one or more unit dosage forms of a second drug wherein said first and second drug are independently selected from the group consisting of non-steroidal anti-inflammatory drugs, proton pump inhibitors, calcium channel blockers, angiotensin converting enzyme (ACE) inhibitors, anti-depressants, selective serotonin reuptake inhibitors, antihistamines, decongestants, biguanides, sulfonylureas, 3-hydroxy-3-methylglutaryl coenzyme A (HMG CoA) reductase inhibitors, anti-epileptic, and anti diabetics; and
administering said first drug and said second drug.

20. The method of claim 19 which provides therapy for 1-28 days.
